(19) United States
(12) Patent Application Publication
(10) Pub. No.: US 2024/0358844 A1
(43) Pub. Date: Oct. 31, 2024

(54) THIOL-BASED MULTIVALENT DRUG DELIVERY COMPOSITIONS

(71) Applicant: ORPRO THERAPEUTICS, INC., San Diego, CA (US)

(72) Inventors: Peter B. HEIFETZ, San Diego, CA (US); Haim MOSKOWITZ, San Diego, CA (US)

(73) Assignee: ORPRO THERAPEUTICS, INC., San Diego, CA (US)

(21) Appl. No.: 18/733,200

(22) Filed: Jun. 4, 2024

Related U.S. Application Data
(63) Continuation of application No. 17/056,151, filed on Nov. 17, 2020.
(60) Provisional application No. 62/672,848, filed on May 17, 2018.

(51) Int. Cl. A61K 47/64; A61K 31/337; A61K 31/407; A61K 31/427; A61K 31/431; A61K 31/475; A61K 31/496; A61K 31/5383; A61K 31/546; A61K 31/665; A61K 31/675; A61K 31/7036; A61K 31/704; A61K 31/7048; A61K 31/7068; A61K 33/243; A61K 38/07; A61K 38/12; A61K 38/13; A61K 38/14

(52) U.S. Cl. CPC A61K 47/64; A61K 31/337; A61K 31/407; A61K 31/427; A61K 31/431; A61K 31/475; A61K 31/496; A61K 31/5383; A61K 31/546; A61K 31/665; A61K 31/675; A61K 31/7036; A61K 31/704; A61K 31/7048; A61K 31/7068; A61K 33/243; A61K 38/07; A61K 38/12; A61K 38/13; A61K 38/14

(57) ABSTRACT

The present invention is related to a drug delivery composition that includes a thioredoxin homologue protein having an N-terminal monocysteinic active site, with the cysteine residue of the active site in a reduced state and an active agent conjugated to the thioredoxin homologue protein and methods of making and using the composition.

### REFERENCE TO RELATED APPLICATIONS

This application claims the benefit of priority under 35 U.S.C. § 119 (e) to U.S. Provisional Patent Application No. 62/672,848, filed May 17, 2018. The entire disclosure of U.S. Provisional Patent Application No. 62/672,848 is incorporated herein by reference.

### GOVERNMENT SUPPORT

This invention was made with Government support under grant number R43HL142395 awarded by the National Institutes of Health (NIH). The Government has certain rights in this invention.

### FIELD OF THE INVENTION

The invention relates to methods and compositions for delivery of active agents to patients by delivery compositions that bind to epithelial surfaces of the body for localized delivery of active agents.

### BACKGROUND OF THE INVENTION

Direct delivery of drugs to mucosal surfaces of the eye, respiratory system, buccal cavity, and gastrointestinal and reproductive tracts by oral, topically-applied or inhaled routes is a powerful strategy to limit potential side effects and toxicity that can result from rapid, high-concentration systemic exposure Mucosal delivery is particularly suitable for inhaled antibiotics which have been used to treat chronic airway infections and have transformed the management of cystic fibrosis (CF) by enabling high drug concentrations at the site of infection while reducing systemic side effects. However, maintaining sufficient epithelial residence time to achieve the desired topical pharmacodynamic effect without reaching toxic systemic concentrations is a key challenge for inhaled therapeutics, many of which are readily taken up across the epithelium. Frequent, low-dose administration creates compliance challenges and may promote antibiotic resistance or anti-drug antibodies (ADA) while larger bolus doses to overcome clearance can result in topical irritation and bronchoconstriction. Tethering drugs to the mucosal surface for sustained release over time has the potential to extend residence at the target site and enhance local bioavailability at safer doses further increasing clinical benefit of inhaled antibiotic therapy in CF.

Ideal mucoadhesive targeting domains (scaffolds) should be able to bind effectively to the epithelial surface, overlying mucus layer or bacterial biofilms, persist long enough to improve residence time of drug payloads vs. free drug bolus, carry multiple drug molecules per scaffold, and be fully biocompatible with minimal risk of reactivity. To date no solutions have effectively addressed these criteria. Early mucoadhesive agents relied on relatively weak, non-specific attachment via hydrogen bonding and hydrophobic or electrostatic interactions. Later improvements introduced targeted covalent interactions with mucus protein cysteines mediated by ‘thiomers’, small-molecule disulfide-binding thiols linked to drug-containing polymeric matrices. However, the advantages conferred by stronger and more specific mucus protein targeting were offset by the potential for inflammation and reactivity posed by introduction of non-physiological polymers (chitosan or polyacrylic) into the sensitive lung environment.

### SUMMARY

One embodiment of the invention is a delivery composition that comprises a thioredoxin homologue protein having an N-terminal monocysteinic active site, with the cysteine residue of the active site in a reduced state. The composition further comprises an active agent conjugated to the thioredoxin homologue protein.

Another embodiment of the invention is a pharmaceutical delivery composition comprising a thioredoxin homologue protein having an N-terminal monocysteinic active site, with the cysteine residue of the active site is in a reduced state, and the composition further comprises an active agent conjugated to the thioredoxin homologue protein. The pharmaceutical delivery composition can be formulated for delivery by a route selected from oral, topical and inhalation

The thioredoxin homologue protein of the compositions of the invention can have a C35S active site. The active agent of the delivery compositions can be conjugated to the thioredoxin homologue protein by a linker that can be a cleavable linker, such as a cleavable ester linker. The linker can be attached to the thioredoxin homologue protein at a lysine residue or multiple lysine residues. The thioredoxin homologue protein can comprises a plurality of linkers, such as more than one linker, more than five linkers or more than ten linkers.

In some embodiments, more than one active agent can be conjugated to the thioredoxin homologue protein, such as more than five active agents or more than ten active agents are conjugated to the thioredoxin homologue protein. The active agent can be selected from a therapeutic active agent, a diagnostic active agent, and an imaging active agent. In embodiments where the delivery composition is conjugated to an active agent that is a therapeutic active agent, the therapeutic active agent can be selected from anti-infectives, radionuclides, chemotherapeutic agents; and cytotoxic agents. When the therapeutic active agent is an anti-infective, it can be selected from vancomycin, tobramycin, amikacin, ciprofloxacin, levofloxacin, colistin, aztreonam, gentamicin, polymyxin B, fosfomycin, ceftazidime, meropenem, carbopenem, imipenem, cefepime, and piperacillin. When the therapeutic active agent is a chemotherapeutic agent, it can be selected from monomethyl auristatin E (MMAE), methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamoxifen, paclitaxel, vincristine, vinblastine, camptothecin, actinomycin D, cytarabine, combrestatin, cyclosporine A, or lifitegrast.

Another embodiment of the invention is a method to treat a condition by delivery of an active agent to an epithelial surface in the body by administering to a patient a composition that includes a thioredoxin homologue protein with an N-terminal monocysteinic active site, with the cysteine residue of the active site in a reduced state. The thioredoxin homologue protein further includes an active agent conjugated to it. The thioredoxin homologue protein can include any embodiments of the thioredoxin homologue protein of the invention. The epithelial surface can be selected from eye, respiratory system, buccal cavity, and gastrointestinal and reproductive tracts of the patient.

A further embodiment of the invention is a method to produce a drug delivery composition that comprises conjugating a thioredoxin homologue protein having an N-terminal monocysteinic active site to an active agent and reducing the cysteine residue of the active site. In one embodiment, fully oxidized monocysteinic thioredoxin homolog protein dimers (thus having blocked active site cysteines) are first formed and then used to initiate the conjugation synthesis. The step of conjugating further comprises reacting the dimers with a linker to produce a linker-conjugated scaffold and conjugating the active agent to the linker to form the drug delivery composition. Once the conjugation reaction is complete, the disulfide bonds of the dimers are reduced, and can be purified.

A still further embodiment of the invention is use of a composition comprising (i) a thioredoxin homologue protein having an N-terminal monocysteinic active site, wherein the cysteine residue of the active site is in a reduced state, and an active agent conjugated to the thioredoxin homologue protein for the treatment of a condition by delivery of an active agent to an epithelial surface in the body.

### BRIEF DESCRIPTION OF THE DRAWINGS

FIG. 1 Demonstration of the ability of glutathione to reduce oxidized thioredoxin and enable reduction of protein disulfide targets. Left side of FIG. 1—polyacrylamide gel electrophoresis of native human thioredoxin-1 (TRX). Lane 1: Molecular weight standards; Lane 2: Oxidized TRX; Lane 3: Oxidized TRX following reaction with dithiothreitol (DTT); Lane 4: Oxidized TRX following reaction with reduced glutathione (GSH); Lane 5: Oxidized TRX following reaction with GSH followed by DTT. Right side of FIG. 1—TRX was oxidized by exposure to ambient air for one week then incubated with either DTT or GSH at pH 8 for 1 hour followed by purification over a size exclusion column to remove unreacted free DTT or GSH after which the TRX was collected and used in a single-turnover insulin protein-disulfide reduction assay. Oxidized TRX (bottom line), TRX reduced with DTT (top line) or TRX reduced with GSH (middle line) were incubated with fully disulfide-bonded heterodimeric insulin at pH 8. After 30 min, 60 min and 120 minutes aliquots were taken and diluted with 0.1% TFA and 17 uM (final) iodoacetic acid and analyzed over RP-HPLC. Absorbance was monitored at 214 nm and the peak area of the insulin heterodimer fraction was determined at each time point to quantify TRX protein disulfide-reducing activity.

FIG. 2 Differential pH dependencies of the thiol reduction states of thioredoxin vs. small-molecule thiol reducing agents Cysteamine, Glutathione, Mesna, and NAC. The fraction of deprotonated thiols over the pH range 6-9 were calculated as 100*(RS-/RSH)/(1+(RS-/RSH) using the Henderson-Hasselbalch equation, pH-pKa+log (RS-/RSH). pH ranges for CF (6.2-6.8) and normal airways (6.8-7.8) and thiol agent pKas were taken from consensus literature values. Mesna, 2-mercaptoethane sulfonate Na; NAC, N-acetyl cysteine.

FIG. 3 Schematic illustration of controlled release of a drug payload conjugated to C35STRX scaffold via cleavable linkers. In this example six vancomycin drug molecules (V) are attached via linkers that decompose at a known rate at different pH. The released vancomycin is able to interact with the cell wall of susceptible pathogens such as Staphylococcus aureus. Attachment of the scaffold: drug conjugate to the epithelial surface is mediated by covalent disulfide bonding.

FIG. 4 Immunohistochemical detection of thioredoxin following IT delivery to rat airways via Penn-Century micro-sprayer. FIG. 4 Top Panel: Reduced (active) C35STRX; FIG. 4 Bottom Panel: Oxidized (inactive) C35STRX. Animals were sacrificed 4 hr post-dose and excised lungs fixed, sectioned and analyzed by IHC for immunodetection of thioredoxin.

FIG. 5A Oxidized C35STRX at room temperature was purified over a 33 mL NAP-5 column. The material in the fraction denoted by the gray box was collected and lyophilized. The lyophilized material was dissolved in 700 μL PBS. A280 was 6.0. Protein concentration was 0.85 mM and the lysine residue concentration was 10 mM.

FIG. 5B 100 mg of NHS-PEG4-Azide was dissolved in 130 μL DMSO to a final concentration of 2 M and 70 μL of this solution was added to 700 μL of C35STRX (in PBS) and the sample was incubated for 2 hours in the dark. After two hours 200 μL of 1M Tris pH-8 was added and the solution was incubated for additional 10 minutes in the dark. The sample was purified over a 33 mL NAP-5 column. Azido-C35STRX (gray boxed region) was collected and lyophilized.

FIG. 5C The lyophilized material was dissolved in 500 μL of 50 mM Tris pH 8.0 and 1 mM EDTA. To this solution 100 μL of 1 M tris pH 9 and 100 μL of 1 M DTT were added. The solution was incubated for 1 hour in the dark and purified over a 33 mL NAP5 column to remove free DTT. The eluted fraction was collected and lyophilized.

FIG. 5D SDS-PAGE analysis of Azido-C35STRX and C35STRX was performed using a 4-12% gradient gel to verify the presence of Azido-C35STRX in the reaction. As shown on the gel the species following reaction with linker (Lane 3) migrates differently from unreacted C35STRX (Lane 2).

FIG. 5E MALDI-TOF analysis of the modified C35STRX indicates that no free C35STRX is present in the sample. The expected mass of unmodified C35STRX is 11673.25 and the measured mass of the sample is 14212, consistent with an average of 11 azido-PEG chains added to each C35STRX molecule.

### DETAILED DESCRIPTION OF THE INVENTION

In one embodiment, the invention provides a delivery composition comprising a thioredoxin homologue protein having a thioredoxin monocysteinic active site, wherein the cysteine residue of the active site corresponds to the N-terminal cysteine of the native thioredoxin active site, which for human thioredoxin-1 is at position 32, and the cysteine residue of the active site is in a reduced state. Such an active site is referred to herein as an “N-terminal monocysteinic active site” or an “N-terminal thioredoxin monocysteinic active site.” The delivery composition further comprises an active agent conjugated to the thioredoxin homologue protein.

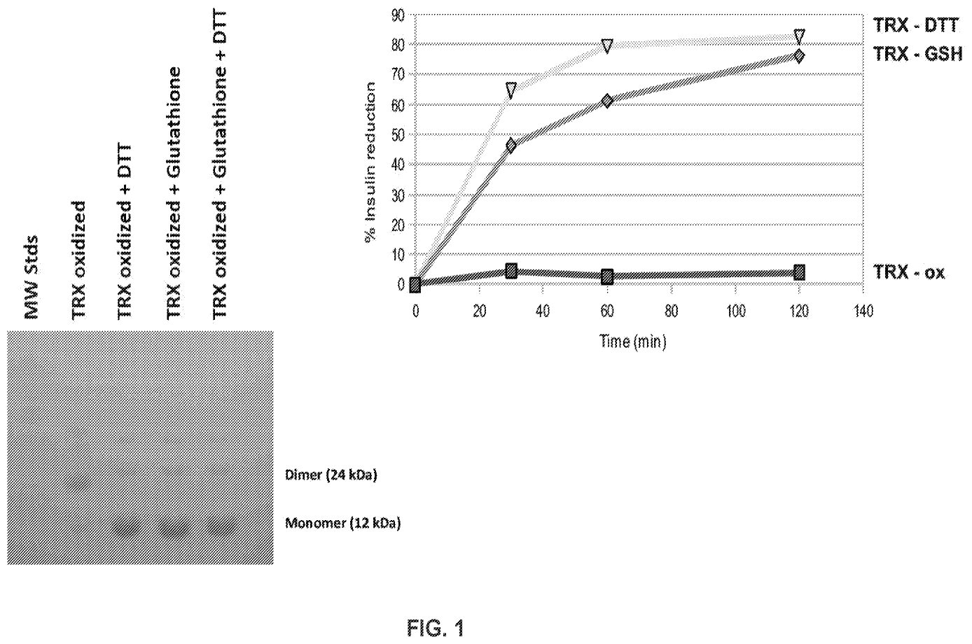

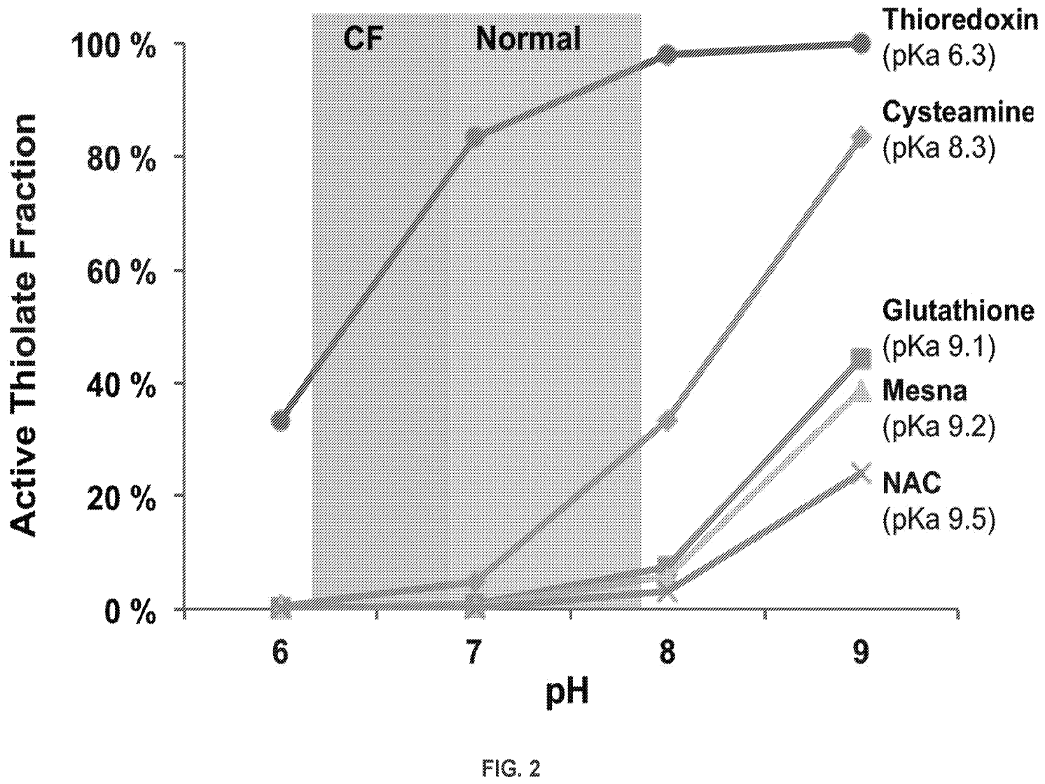

The thioredoxin homologue protein of the invention is a mucus-targeting scaffold based on a monothiol (or monocysteinic) active site variant of the endogenously secreted human epithelial protein thioredoxin-1 (TRX) (e.g., C35STRX) modified to bind covalently to soluble and membrane-associated mucus proteins. TRX is a small (12 kDa) redox enzyme that regulates protein and enzyme activity via potent and selective disulfide bond reduction mediated by a highly conserved dithiol Cys-Gly-Pro-Cys active site. Extracellular TRX has anti-inflammatory properties and is required for activity of the potent mucosal antimicrobial beta-defensin. Reductive activation of oxidized TRX normally proceeds via the flavoenzyme thioredoxin reductase (TRXR) and the cofactor NADPH, but as shown in FIG. 1 TRX can also be reduced and recycled in the absence of TRXR by reduced glutathione (GSH), a tripeptide reductant abundant in the human airway. Extracellular TRX is localized to both the airway surface liquid layer and the submucosal glands and comprises a significant proportion of the epithelial proteins that are secreted along with newly formed airway mucus. At relative GSH: TRX concentrations comparable to that in the airway, GSH is capable of restoring insulin disulfide bond reduction activity to oxidized TRX (FIG. 1). Thus, extracellular TRX may function homeostatically in normal airways to prevent excess disulfide bonds in mucus proteins. There are nearly 300 Cys per mucin monomer (UniProt accessions P98088 and Q9HC84) yet only 5% of these residues are disulfide-bonded in normal individuals vs. 30% or more in CF, associated with pathologic increases in mucus viscosity and stiffness (Yuan et al., 2015, Science Translational Med 7, 276ra227). TRX is an unusually potent normalizer of CF sputum viscoelasticity due in part to the unusually acidic acid-dissociation constant (pKa) of its catalytic Cys thiol which, at pH 6.2, is three logs lower than that of GSH (FIG. 2). Hence, GSH is unable to effectively support TRX cycling in the abnormally acidic pH of the CF airway, as the formation of chemically active GSH thiolate anions is markedly attenuated at pH levels two logs or more below its pKa.

Reduced N-terminal monocysteinic TRX, like native TRX, can act to thin excessively viscoelastic CF secretions and restore normal rates of mucociliary transport on epithelial surfaces in situ. As a monothiol, N-terminal monocysteinic TRX lacks the ability to resolve mixed-disulfides and unlike native TRX stays bound covalently to targeted disulfide bonds. This crucially improves its utility as a drug by prolonging activity via long-duration Cys blockade, coupled with attenuated systemic uptake and hence low toxicity due to sequestration in the mucus layer. Because of this stoichiometric mechanism, N-terminal monocysteinic TRX can be delivered in a fully-reduced form that does not require cofactors and is independent of airway GSH activity. The lack of observed toxic effects in normal animals dosed by aerosol at many times the anticipated 40 mg per day CF clinical dose suggests that in addition to viscosity-normalization properties, N-terminal monocysteinic TRX may also be a safe and effective means of covalent attachment to epithelial mucus in humans. Linking up to 12 drug payload molecules to each N-terminal monocysteinic TRX is contemplated herein and experiments have shown that conjugation to a model payload (biotin) does not disrupt N-terminal monocysteinic TRX mucoadhesion.

An “N-terminal monocysteinic active site” of the present invention comprises the amino acid sequence C-X-X-X (SEQ ID NO:17) (native or wild-type sequence comprises the amino acid sequence C-X-X-C having SEQ ID NO:16). As used herein, amino acid residues denoted “C” are cysteine residues and amino acid residues denoted “X” can be any amino acid residue other than a cysteine residue, and in particular, any of the remaining standard 20 amino acid residues. Such an N-terminal monocysteinic active site of the present invention preferably comprises the amino acid sequence C-G-P-X (SEQ ID NO: 18), wherein the native or wild-type sequence comprises the amino acid sequence C-G-P-C(SEQ ID NO: 1). An N-terminal monocysteinic active site can further comprise the amino acid sequence X-C-X-X-X-X (SEQ ID NO:19), wherein the native or wild-type sequence comprises the amino acid sequence X-C-X-X-C-X (SEQ ID NO:20). Preferably, an N-terminal monocysteinic active site of the present invention comprises the amino acid sequence X-C-G-P-X-X (SEQ ID NO:21), wherein such amino acid residue denoted “G” is a glycine residue, and wherein such amino acid residue denoted “P” is a proline residue, wherein the native or wild-type sequence comprises the amino acid sequence X-C-G-P-C-X (SEQ ID NO:22). More preferably, an N-terminal monocysteinic active site of the present invention comprises the amino acid sequence W-C-G-P-X-K (SEQ ID NO:23), wherein such amino acid residue denoted “W” is a tryptophan residue, and wherein such amino acid residue denoted “K” is a lysine residue and wherein the native sequence comprises the amino acid sequence W-C-G-P-C-K (SEQ ID NO:3). Preferably, an N-terminal monocysteinic active site can comprise the amino acid sequence C-X-X-S(SEQ ID NO:24). Such an N-terminal monocysteinic active site of the present invention preferably comprises the amino acid sequence C-G-P-S(SEQ ID NO:1). An N-terminal monocysteinic active site can further comprise the amino acid sequence X-C-X-X-S-X (SEQ ID NO:25), X-C-G-P-S-X (SEQ ID NO: 26) or W-C-G-P-S-K (SEQ ID NO:27), wherein amino acid residues denoted “X” can be any amino acid residue other than a cysteine residue. Reference to “thioredoxin active site” includes N-terminal monocysteinic active sites and native or wildtype thioredoxin active sites.

In one aspect of the invention, the protein containing an N-terminal monocysteinic active site is a full-length thioredoxin protein or any fragment thereof containing an N-terminal monocysteinic active site as described structurally and functionally above. Preferred modified thioredoxin proteins having N-terminal monocysteinic active sites include prokaryotic thioredoxin, yeast thioredoxin, plant thioredoxin, and mammalian thioredoxin, with human thioredoxin being particularly preferred. The nucleic acid and amino acid sequences of thioredoxins from a variety of organisms are well known in the art and are intended to be encompassed by the present invention. For example, SEQ ID NOs: 4-15 represent the amino acid sequences for thioredoxin from Pseudomonas syringae (SEQ ID NO: 4), Porphyromonas gingivalis (SEQ ID NO:5), Listeria monocytogenes (SEQ ID NO: 6), Saccharomyces cerevisiae (SEQ ID NO:7), Gallus gallus (SEQ ID NO:8), Mus musculus (SEQ ID NO:9), Rattus norvegicus (SEQ ID NO: 10), Bos taurus (SEQ ID NO:11), Homo sapiens (SEQ ID NO: 12), Arabidopsis thaliana (SEQ ID NO: 13), Zea mays (SEQ ID NO: 14), and Oryza sativa (SEQ ID NO:15). Referring to each of these sequences, the X-C-G-P-C-X (SEQ ID NO:22) motif (which includes the CGPC motif of SEQ ID NO:1) can be found as follows: SEQ ID NO:4 (positions 33-38), SEQ ID NO:5 (positions 28-33), SEQ ID NO: 6 (positions 27-32), SEQ ID NO: 7 (positions 29-34), SEQ ID NO:8 (positions 31-36), SEQ ID NO:9 (positions 31-36), SEQ ID NO:10 (positions 31-36), SEQ ID NO:11 (positions 31-36), SEQ ID NO: 12 (positions 31-36), SEQ ID NO: 13 (positions 59-64), SEQ ID NO: 14 (positions 88-93) and SEQ ID NO:15 (positions 94-99). Moreover, the three-dimensional structure of several thioredoxin proteins has been resolved, including human and bacterial thioredoxins. Therefore, the structure and active site of thioredoxins from multiple organisms is well known in the art and one of skill in the art would be able to readily identify and produce fragments or homologues of full-length thioredoxins, including thioredoxins having N-terminal monocysteinic active sites that can be used in the present invention.

The phrase “in a reduced state” specifically describes the state of the cysteine residues in the active site of a protein or peptide of the present invention. In a reduced state, adjacent cysteine residues form a dithiol (i.e. two free sulfhydryl groups, —SH). In contrast, in oxidized form, such cysteine residues form an intramolecular disulfide bridge; such a molecule can be referred to as cystine. In a reduced state, an N-terminal monocysteinic active site is capable of participating in redox reactions through the reversible oxidation of its active site thiol to a disulfide, and catalyzes thiol-disulfide exchange reactions that result in covalent linkage to one of the target disulfide Cys. For proteins or peptides of the present invention containing an N-terminal monocysteinic active site, the N-terminal cysteine in the active site is in a reduced state as a monothiol and is therefore able to form a stable mixed-disulfide with a cysteine on the target protein.

As used herein, a protein of the present invention containing an N-terminal monocysteinic active site can be an N-terminal monocysteinic active site per se or an N-terminal monocysteinic active site joined to other amino acids by glycosidic linkages. Thus, the minimal size of a protein or peptide of the present invention is from about 4 to about 6 amino acids in length, with preferred sizes depending on whether a full-length, fusion, multivalent, or merely functional portions of such a protein is desired. Preferably, the length of a protein or peptide of the present invention extends from about 4 to about 100 amino acid residues or more, with peptides of any interim length, in whole integers (i.e., 4, 5, 6, 7 . . . 99, 100, 101 . . . ), being specifically envisioned. It may also be a short thioredoxin mimetic peptide blocked at the N and C termini as described by Bachnoff et al., Free Radical Biol Med 50:1355-67, 2011. In a further preferred embodiment, a protein of the present invention can be a full-length protein or any homologue of such a protein. As used herein, the term “homologue” is used to refer to a protein or peptide which differs from a naturally occurring protein or peptide (i.e., the “prototype” or “wildtype” protein) by modifications to the naturally-occurring protein or peptide, but which maintains the basic protein and side chain structure of the naturally-occurring form, and/or which maintains a basic three-dimensional structure of at least a biologically active portion (e.g., the thioredoxin active site) of the native protein. Such changes include, but are not limited to: changes in one or a few amino acid side chains; changes in one or a few amino acids, including deletions (e.g., a truncated version of the protein or peptide (fragment)), insertions and/or substitutions; changes in stereochemistry of one or a few atoms; and/or minor derivatizations, including but not limited to: methylation, glycosylation, phosphorylation, acetylation, myristoylation, prenylation, palmitoylation, amidation and/or addition of glycosylphosphatidyl inositol. According to the present invention, any protein or peptide useful in the present invention, including homologues of natural thioredoxin proteins, have an N-terminal monocysteinic active site such that, in a reduced state, the protein or peptide is capable of participating in redox reactions through the oxidation of its active site thiol to a disulfide and/or of decreasing the viscosity or cohesiveness of mucus or sputum or increasing the liquefaction of mucus or sputum. As used herein, a protein or peptide containing an N-terminal monocysteinic active site can have characteristics similar to thioredoxin, and preferably, is a thioredoxin selected from the group of prokaryotic thioredoxin, fungal thioredoxin (including yeast), plant thioredoxin, animal thioredoxin, avian thioredoxin, or mammalian thioredoxin. In a particularly preferred embodiment, the protein is human thioredoxin.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in homologues, as compared to the wild-type protein, either agonize, antagonize, or do not substantially change, the basic biological activity of the homologue as compared to the naturally occurring protein. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally-occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

In one embodiment, proteins or peptides containing an N-terminal monocysteinic active site can be products of drug design or selection and can be produced using various methods known in the art. Such proteins or peptides can be referred to as mimetics. A mimetic refers to any peptide or non-peptide compound that is able to mimic the biological action of a naturally-occurring peptide, often because the mimetic has a basic structure that mimics the basic structure of the naturally-occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety. Thioredoxin mimetic peptides capable of potent and selective redox activity are described by Bachnoff et al., Free Radical Biol Med 50:1355-67 (2011) and incorporated herein by reference in its entirety.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Diversity-creation methods such as the foregoing can be combined with other techniques designed to improve function or pharmacology, especially for reduced-size molecules like active-site mimetics. For example, one approach that has shown promise in early-stage studies is hydrocarbon-stapled a-helical peptides, a novel class of synthetic miniproteins locked into their bioactive a-helical fold through the site-specific introduction of a chemical brace, an all-hydrocarbon staple. Stapling can greatly improve the pharmacologic performance of peptides, increasing their target affinity and proteolytic resistance, while creating smaller peptide versions of larger proteins/enzymes that are suitable for chemical synthesis (Verdine, G. L. and Hilinsky, G. J., Methods Enzymol, 503:3-33, 2012).

In one embodiment of the present invention, a protein suitable for use in the present invention has an amino acid sequence that comprises, consists essentially of, or consists of a full length sequence of a thioredoxin protein or any fragment thereof that has an N-terminal monocysteinic active site as described herein. For example, any one of the native sequences of SEQ ID NOs 4-15 or a fragment or other homologue thereof that contains an N-terminal monocysteinic active site as described herein is encompassed by the invention. Such homologues can include proteins having an amino acid sequence that is at least about 10% identical to the amino acid sequence of a full-length thioredoxin protein, or at least 20% identical, or at least 30% identical, or at least 40% identical, or at least 50% identical, or at least 60% identical, or at least 70% identical, or at least 80% identical, or at least 90% identical, or greater than 95% identical to the amino acid sequence of a full-length thioredoxin protein, including any percentage between 10% and 100%, in whole integers (10%, 11%, 12%, . . . 98%, 99%, 100%).

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:
Reward for match=1
Penalty for mismatch=−2
Open gap (5) and extension gap (2) penalties
gap x_dropoff (50) expect (10) word size (11) filter (on)
   For blastp, using 0 BLOSUM62 matrix:
Open gap (11) and extension gap (1) penalties
gap x_dropoff (50) expect (10) word size (3) filter (on).

A protein useful in the present invention can also include proteins having an amino acid sequence comprising at least 10 contiguous amino acid residues of any full-length thioredoxin protein containing an N-terminal monocysteinic active site (native sequences represented by SEQ ID NOs: 4-15, i.e., 10 contiguous amino acid residues having 100% identity with 10 contiguous amino acids of a reference sequence). In other embodiments, a homologue of a thioredoxin protein includes amino acid sequences comprising at least 15, or at least 20, or at least 25, or at least 30, or at least 35, or at least 40, or at least 45, or at least 50, or at least 55, or at least 60, or at least 65, or at least 70, or at least 75, or at least 80 contiguous amino acid residues of the amino acid sequence of a naturally occurring thioredoxin protein, and so on, up to the full-length of the protein, including any intervening length in whole integers (10, 11, 12, . . . ) and which comprises an N-terminal monocysteinic active site.

According to the present invention, the term “contiguous” or “consecutive”, with regard to sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, a protein useful in the present invention includes a protein having an amino acid sequence that is sufficiently similar to a natural thioredoxin amino acid sequence that a nucleic acid sequence encoding the homologue is capable of hybridizing under moderate, high or very high stringency conditions (described below) to (i.e., with) a nucleic acid molecule encoding the natural thioredoxin protein (i.e., to the complement of the nucleic acid strand encoding the natural thioredoxin amino acid sequence). Such hybridization conditions are described in detail below.

A nucleic acid sequence complement of nucleic acid sequence encoding a thioredoxin protein of the present invention refers to the nucleic acid sequence of the nucleic acid strand that is complementary to the strand that encodes thioredoxin. It will be appreciated that a double-stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence of a thioredoxin protein, and/or with the complement of the nucleic acid sequence that encodes such amino acid sequence. Methods to deduce a complementary sequence are known to those skilled in the art.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA: RNA or DNA: DNA hybrids are being formed. Calculated melting temperatures for DNA: DNA hybrids are 10° C. less than for DNA: RNA hybrids. In particular embodiments, stringent hybridization conditions for DNA: DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA: RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA: DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

A protein of the present invention can also be a fusion protein that includes a segment containing an N-terminal monocysteinic active site and a fusion segment that can have a variety of functions. For example, such a fusion segment can function as a tool to simplify purification of a protein of the present invention, such as to enable purification of the resultant fusion protein using affinity chromatography. A suitable fusion segment can be a domain of any size that has the desired function (e.g., imparts increased stability to a protein, imparts increased immunogenicity to a protein, and/or simplifies purification of a protein). It is within the scope of the present invention to use one or more fusion segments. Fusion segments can be joined to amino and/or carboxyl termini of the segment containing an N-terminal monocysteinic active site. Linkages between fusion segments and thioredoxin active site-containing domains of fusion proteins can be susceptible to cleavage in order to enable straightforward recovery of the thioredoxin monocysteinic active site-containing domains of such proteins. Fusion proteins are preferably produced by culturing a recombinant cell transformed with a fusion nucleic acid molecule that encodes a protein including the fusion segment attached to either the carboxyl and/or amino terminal end of an N-terminal monocysteinic active site-containing domain.

In one embodiment, a protein or peptide containing an N-terminal monocysteinic active site suitable for use with the method of the present invention comprises a protein or peptide containing an N-terminal monocysteinic active site derived from a substantially similar species of animal as that to which the protein is to be administered. In another embodiment, any protein or peptide containing an N-terminal monocysteinic active site, including from diverse sources such as microbial, plant and fungus can be used in a given patient.

In one embodiment of the present invention, any of the amino acid sequences described herein, such as the amino acid sequence of a naturally occurring thioredoxin protein or thioredoxin containing a monocysteinic active site, can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally-occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

In another embodiment, a protein or peptide containing an N-terminal monocysteinic active site suitable for use with the method of the present invention comprises an isolated, or biologically pure, protein. As such, “isolated” and “biologically pure” do not necessarily reflect the extent to which the protein has been purified. An isolated protein of the present invention can, for example, be obtained from its natural source, be produced using recombinant DNA technology (e.g., polymerase chain reaction (PCR) amplification, cloning), or be synthesized chemically.

In yet another embodiment, a chemically-synthetic protein or peptide containing an N-terminal monocysteinic active site of the present invention may also refer to a stabilized version, such as one containing an active site constrained structurally by stapled peptide technology, by cyclization, or by constraint at the N or C termini. Preferably, the protein containing an N-terminal monocysteinic active site to be used in methods of the invention have a half-life in vivo that is sufficient to cause a measurable or detectable increase in liquefaction (or decrease in the viscoelasticity or cohesiveness) of mucus or sputum in a patient, and or to cause a measurable, detectable or perceived therapeutic benefit to the patient that is associated with the mucus and sputum in the patient. Such half-life can be effected by the method of delivery of such a protein. A protein of the present invention preferably has a half-life of greater than about 5 minutes in an animal, and more preferably greater than about 4 hours in an animal, and even more preferably greater than about 16 hours in an animal. In a preferred embodiment, a protein of the present invention has a half-life of between about 5 minutes and about 24 hours in an animal, and preferably between about 2 hours and about 16 hours in an animal, and more preferably between about 4 hours and about 12 hours in an animal.

Further embodiments of the present invention include nucleic acid molecules that encode a protein or peptide containing an N-terminal monocysteinic active site. Such nucleic acid molecules can be used to produce a protein that is useful in the method of the present invention in vitro or in vivo. A nucleic acid molecule of the present invention includes a nucleic acid molecule comprising, consisting essentially of, or consisting of, a nucleic acid sequence encoding any of the proteins described previously herein. In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode the desired protein of the present invention or to form stable hybrids under stringent conditions with natural gene isolates. An isolated nucleic acid molecule can include degeneracies. As used herein, nucleotide degeneracies refers to the phenomenon that one amino acid can be encoded by different nucleotide codons. Thus, the nucleic acid sequence of a nucleic acid molecule that encodes a given protein useful in the present invention can vary due to degeneracies.

According to the present invention, reference to a gene includes all nucleic acid sequences related to a natural (i.e. wildtype) gene as well as those related to the thioredoxin monocysteinic active site, such as regulatory regions that control production of the protein encoded by that gene (such as, but not limited to, transcription, translation or post-translation control regions) as well as the coding region itself. In another embodiment, a gene can be a naturally occurring allelic variant that includes a similar but not identical sequence to the nucleic acid sequence encoding a given protein. Allelic variants have been previously described above. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

A nucleic acid molecule homologue can be produced using a number of methods known to those skilled in the art (e.g., as described in Sambrook et al., ibid). For example, nucleic acid molecules can be modified using a variety of techniques including, but not limited to, by classical mutagenesis and recombinant DNA techniques (including without limitation site-directed mutagenesis, chemical treatment, restriction enzyme cleavage, ligation of nucleic acid fragments and/or PCR amplification), or synthesis of oligonucleotide mixtures and chemical ligation, or in vitro or in vivo recombination, of mixtures of molecular groups to “build” a re-assorted library of nucleic acid molecules comprising a multiplicity of combinations thereof by the process of gene shuffling (i.e., molecular breeding; see, for example, U.S. Pat. No. 5,605,793 to Stemmer; Minshull and Stemmer, Curr. Opin. Chem. Biol. 3:284-290, 1999; Stemmer, P.N.A.S. USA 91:10747-10751, 1994, all of which are incorporated herein by reference in their entirety). These and other similar techniques known to those skilled in the art can be used to efficiently introduce multiple simultaneous changes in the protein. Nucleic acid molecule homologues can subsequently be selected by hybridization with a given gene, or be screened by expression directly for function and biological activity of proteins encoded by such nucleic acid molecules.

One embodiment of the present invention relates to a recombinant nucleic acid molecule that comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a replicating plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the protein containing an N-terminal monocysteinic active site) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector that enable the transcription and translation of the nucleic acid sequence within the recombinant host cell.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Typically, a recombinant nucleic acid molecule includes at least one nucleic acid molecule of the present invention operatively linked to one or more expression control sequences. As used herein, the phrase “recombinant molecule” or “recombinant nucleic acid molecule” refers primarily to a nucleic acid molecule or nucleic acid sequence operatively linked to an expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule is able to be expressed when transfected (i.e., transformed, transduced, transfected, conjugated or conduced) into a host cell. Transcription control sequences are expression control sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced. Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those that are integrated into the host cell chromosome, also contains secretory signals (i.e., signal-segment or signal-sequence nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Other signal sequences include those capable of directing periplasmic or extracellular secretion, or retention within desired compartments. Suitable leader sequences include a leader sequence that is naturally associated with the protein, or any heterologous leader sequence capable of directing the delivery and insertion of the protein to the membrane of a cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants. In microbial systems, the term “transformation” is used to describe an inherited change due to the acquisition of exogenous nucleic acids by the microorganism and is essentially synonymous with the term “transfection.” However, in animal cells, transformation has acquired a second meaning which can refer to changes in the growth properties of cells in culture (described above) after they become cancerous, for example. Therefore, to avoid confusion, the term “transfection” is preferably used with regard to the introduction of exogenous nucleic acids into animal cells, and is used herein to generally encompass transfection of animal cells and transformation of plant cells and microbial cells, to the extent that the terms pertain to the introduction of exogenous nucleic acids into a cell. Therefore, transfection techniques include, but are not limited to, transformation, particle bombardment, electroporation, microinjection, lipofection, adsorption, infection and protoplast fusion.

The thioredoxin homologue protein with an N-terminal monocysteinic active site of the invention is further characterized as having an active agent conjugated to the thioredoxin homologue protein. The active agent can be a therapeutic agent (including a chemotherapeutical agent), a diagnostic agent, or an imaging agent. In some embodiments the active agent is a small molecule, radionuclide, peptide, peptidomimetic, protein, antisense oligonucleotide, peptide nucleic acid, siRNA, metal chelate, or carbohydrate.

“Active agent” as used herein may be any suitable active agent, including therapeutic, diagnostic or imaging agents.

“Therapeutic agent” as used herein may be any suitable therapeutic agent, including but not limited to anti-infectives, radionuclides, chemotherapeutic agents; and cytotoxic agents. More particularly, suitable therapeutic agents can be selected from parathyroid hormone related protein (parathyroid hormone related protein), growth hormone (GH) particularly human and bovine growth hormone, growth hormone-releasing hormones, interferon including α-, β-, or γ-interferons, etc., interleukin-I, interleukin-II, erythropoietin including α- and β-erythropoietin (EPO), granulocyte colony stimulating factor (GCSF), granulocyte macrophage colony stimulating factor (GM-CSF), anti-angiogenic proteins (e.g., angiostatin, endostatin) PACAP polypeptide (pituitary adenylate cyclase activating polypeptide), vasoactive intestinal peptide (VIP), thyrotrophin releasing hormone (TRH), corticotrophin releasing hormone (CRH), vasopressin, arginine vasopressin (AVP), angiotensin, calcitonin, atrial naturetic factor, somatostatin, adrenocorticotropin, gonadotropin releasing hormone, oxytocin, insulin, somatotropin, HBS antigen of hepatitis B virus, plasminogen tissue activator, coagulation factors including coagulation factors VIII and IX, glucosylceramidase, sargramostim, lenograstim, filgrastim, interleukin-2, dornase-alpha., molgramostim, PEG-L-asparaginase, PEG-adenosine deaminase, hirudin, eptacog-α (human blood coagulation factor VIIa) nerve growth factors, transforming growth factor, epidermal growth factor, basic fibroblast growth factor, VEGF, heparin including low molecular weight heparin, calcitonin, atrial naturetic factor, antigens, somatostatin, adrenocorticotropin, gonadotropin releasing hormone, oxytocin, vasopressin, cromolyn sodium, vancomycin, desferrioxamine (DFO), parathyroid hormone, anti-cholinergics, cyclosporines including cyclosporine A, lifitegrast, gallium, anti-inflammatoires, antimicrobials, antifungals, an immunogen or antigen, an antibody such as a monoclonal antibody, or any combination thereof. See, e.g., U.S. Pat. Nos. 6,967,028; 6,930,090; and 6,972,300.

Example therapeutic agents include all of the therapeutic agents set forth in paragraphs 0065 through 0388 of W. Hunter, D. Gravett, et al., US Patent Application Publication No. 20050181977 (Published Aug. 18, 2005) (assigned to Angiotech International AG) the disclosure of which is incorporated by reference herein in its entirety.

“Anti-infective” as described herein can be any anti-infective agent suitable for preventing, treating, or curing infection by an infectious agent, including but not limited to amebicides, aminoglycosides, anthelmintics, antifungals (such as azole antifungals, echinocandins, miscellaneous antifungals, polyenes), antimalarial agents, antimalarial combinations (such as antimalarial quinolines), antituberculosis agents, (such as aminosalicylates, antituberculosis combinations, diarylquinolines, hydrazide derivatives, miscellaneous antituberculosis agents, nicotinic acid derivatives, rifamycin derivatives, streptomyces derivatives), antiviral agents (such as adamantane antivirals, antiviral boosters, antiviral combinations, antiviral interferons, chemokine receptor antagonist, integrase strand transfer inhibitor, miscellaneous antivirals, neuraminidase inhibitors, NNRTIs, NS5A inhibitors, nucleoside reverse transcriptase inhibitors (NRTIs), protease inhibitors, purine nucleosides), carbapenems, carbapenems/beta-lactamase inhibitors, cephalosporins (such as cephalosporins/beta-lactamase inhibitors, first generation cephalosporins, fourth generation cephalosporins, next generation cephalosporins, second generation cephalosporins, third generation cephalosporins), glycopeptide antibiotics, glycylcyclines, leprostatics, lincomycin derivatives, macrolide derivatives (such as ketolides, macrolides), antibiotics (such as vancomycin, tobramycin, amikacin, ciprofloxacin, levofloxacin, colistin, aztreonam, gentamicin, polymyxin B, fosfomycin, ceftazidime, meropenem, carbopenem, imipenem, cefepime, or piperacillin), oxazolidinone antibiotics, penicillins, (such as aminopenicillins, antipseudomonal penicillins, beta-lactamase inhibitors, natural penicillins, penicillinase resistant penicillins), quinolones, streptogramins, sulfonamides, tetracyclines, and urinary anti-infectives.

“Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ¹¹³mIn, ¹¹⁵ mIn, ¹²³I, ¹²⁵I, ¹³¹I, ¹⁸⁹Ir, ¹⁹¹Ir, ¹⁹²Ir, ¹⁹⁴Ir, ⁵²Fc, ⁵⁵Fc, ⁵⁹Fc, ¹⁷⁷Lu, ¹⁰⁹Pd, ³²P, ²²⁶Ra, ¹⁸⁶Rc, ¹⁸⁸Rc, ¹⁵³Sm, ⁴⁶Sc, ⁴⁷Sc, ⁷²Sc, ⁷⁵Sc, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ¹⁰¹mRh, and ²¹²Pb. Radionuclides may also be those useful for delivering a detectable dosage for imaging or diagnostic purposes, even where those compounds are not useful for therapeutic purposes.

“Chemotherapeutic agent” as used herein includes but is not limited to monomethyl auristatin E (MMAE), methotrexate, daunomycin, mitomycin, cisplatin (cisplatinum or cis-dianminedichloroplatinum (II) (CCDP)), vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamoxifen, paclitaxel, vincristine, vinblastine, camptothecin, actinomycin D, cytarabine, combrestatin and its derivatives.

“Cytotoxic agent” as used herein includes but is not limited to ricin (or more particularly the ricin A chain), aclacinomycin, diphtheria toxin, Monensin, Verrucarin A, Abrin, Vinca alkaloids, Tricothecenes, and Pseudomonas exotoxin A.

“Immunogen” and “antigen” are used interchangeably and mean any compound to which a cellular or humoral immune response is to be directed against, and include bacterial antigens, viral antigens, and tumor antigens. Non-living immunogens (e.g., killed immunogens, subunit vaccines, recombinant proteins or peptides or the like) are currently preferred. Examples of suitable immunogens include those derived from bacterial surface polysaccharides which can be used in carbohydrate-based vaccines. Bacteria typically express carbohydrates on their cell surface as part of glycoproteins, glycolipids, O-specific side chains of lipopolysaccharides, capsular polysaccharides and the like. Exemplary bacterial strains include Streptococcus pneumonia, Neisseria meningitidis, Haemophilus influenza, Klebsiella spp., Pseudomonas spp., Salmonella spp., Shigella spp., and Group B streptococci. A number of suitable bacterial carbohydrate epitopes which may be used as the immunogen in the present invention are described in the art (e.g., Sanders, et al. Pediatr. Res. 37:812-819 (1995); Bartoloni, et al. Vaccine 13:463-470 (1995); Pirofski, et al., Infect. Immun. 63:2906-2911 (1995) and International Publication No. WO 93/21948) and are further described in U.S. Pat. No. 6,413,935. Exemplary viral antigen or immunogen includes those derived from HIV (e.g., gp120, nef, tat, pol). Exemplary fungal antigens include those derived from Candida albicans, Cryptococcus neoformans, Coccidoides spp., Histoplasma spp., and Aspergillus spp. Parasitic antigens include those derived from Plasmodium spp., Trypanosoma spp., Schistosoma spp., Leishmania spp. and the like. Exemplary carbohydrate epitopes that may be utilized as antigens or immunogens in the present invention include but are not limited to the following: Galα1,4Galβ-(for bacterial vaccines); GalNAcα-(for cancer vaccines); Manβ1,2 (Manβ) nManβ-(for fungal vaccines useful against, for example, Candida albicans); GalNAcβ1,4 (NeuAcα2,3) Galβ1,4Glcβ-O-ceramide (for cancer vaccines); Galα1,2 (Tyvα1,3) Manα1,4Rhaα1,3Galα1,2 (Tyaα1,3) Manα4Rha- and Galα1,2 (Abeα1,3) Manα1,4Rhaα1,3Galα1,2 (Abeα1,3) Manα1,4Rhaα1,3Galα1,2 (Abeα1,3) Manα1,4Rha-(both of which are useful against, for example, Salmonella spp.). Carbohydrate epitopes as antigens or immunogens and the synthesis thereof are described further in U.S. Pat. No. 6,413,935. In one embodiment the immunogen may be an anthrax immunogen; i.e. an immunogen that produces protective immunity to Bacillus anthracis, such as anthrax vaccine, A, (Michigan Department of Health, Lansing, Mich.; described in U.S. Pat. No. 5,728,385). Other examples of immunogens or antigens include but are not limited to those that produce an immune response or antigenic response to the following diseases and disease-causing agents: adenoviruses; Bordetella pertussus; Botulism; bovine rhinotracheitis; Branhamella catarrhalis; canine hepatitis; canine distemper; Chlamydiae; Cholera; coccidiomycosis; cowpox; cytomegalovirus; cytomegalovirus; Dengue fever; dengue toxoplasmosis; Diphtheria; encephalitis; Enterotoxigenic Escherichia coli; Epstein Barr virus; equine encephalitis; equine infectious anemia; equine influenza; equine pneumonia; equine rhinovirus; feline leukemia; flavivirus; Globulin; haemophilus influenza type b; Haemophilus influenzae; Haemophilus pertussis; Helicobacter pylori; Hemophilus; hepatitis; hepatitis A; hepatitis B; Hepatitis C; herpes viruses; HIV; HIV-1 viruses; HIV-2 viruses; HTLV; Influenza; Japanese encephalitis; Klebsiellae species; Legionella pneumophila; leishmania; leprosy; lyme disease; malaria immunogen; measles; meningitis; meningococcal; Meningococcal Polysaccharide Group A; Meningococcal Polysaccharide Group C; mumps; Mumps Virus; mycobacteria and; Mycobacterium tuberculosis; Neisseria; Neisseria gonorrhoeae; Neisseria meningitidis; ovine blue tongue; ovine encephalitis; papilloma; parainfluenza; paramyxovirus; paramyxoviruses; Pertussis; Plague; Pneumococcus; Pneumocystis carinii; Pneumonia; Poliovirus; Proteus species; Pseudomonas aeruginosa; rabies; respiratory syncytial virus; rotavirus; Rubella; Salmonellae; schistosomiasis; Shigellae; simian immunodeficiency virus; Smallpox; Staphylococcus aureus; Staphylococcus species; Streptococcus pneumoniae; Streptococcus pyogenes; Streptococcus species; swine influenza; tetanus; Treponema pallidum; Typhoid; Vaccinia; varicella-zoster virus; and Vibrio cholerae. The antigens or immunogens may, include various toxoids, viral antigens and/or bacterial antigens such as antigens commonly employed in the following vaccines: chickenpox vaccine; diphtheria, tetanus, and pertussis vaccines; Haemophilus influenzae type b vaccine (Hib); hepatitis A vaccine; hepatitis B vaccine; influenza vaccine; measles, mumps, and rubella vaccines (MMR); pneumococcal vaccine; polio vaccines; rotavirus vaccine; anthrax vaccines; and tetanus and diphtheria vaccine (Td). See, e.g., U.S. Pat. No. 6,309,633. Antigens or immunogens that are used to carry out the present invention include those that are derivatized or modified in some way, such as by conjugating or coupling one or more additional groups thereto to enhance function or achieve additional functions such as targeting or enhanced delivery thereof, including but not limited to those techniques described in U.S. Pat. No. 6,493,402 to Pizzo et al. (α-2 macroglobulin complexes); U.S. Pat. Nos. 6,309,633; 6,207,157; 5,908,629, etc.

Interferon (IFNs) are used herein refers to natural proteins produced by the cells of the immune system of most vertebrates in response to challenges by foreign agents such as viruses, bacteria, parasites and tumor cells, and its function is to inhibit viral replication within other cells. Interferons belong to the large class of glycoproteins known as cytokines Three major classes of interferons for human have been discovered as type I, type II and type III, classified according to the type of receptor through which they signal. Human type I IFNs comprise a vast and growing group of IFN proteins, designated IFN-α, IFN-β, IFN-κ, IFN-δ, IFN-ε, IFN-τ, IFN-ω and IFN-ξ. [See Interferon-ξ/limitin: Novel type I Interferon that displays a narrow range of biological activity, Oritani Kenji and Tomiyama Yoshiaki, International Journal of hematology, 2004, 80, 325-331; Characterization of the type I interferon locus and identification of novel genes, Hardy et al., Genomics, 2004, 84, 331-345.] Homologous molecules to type I IFNs are found in many species, including most mammals, and some have been identified in birds, reptiles, amphibians and fish species. [See The interferon system of non-mammalian vertebrates, Schultz et al., Developmental and Comparative Immunology, 28, 499-508.] All type I IFNs bind to a specific cell surface receptor complex known as the IFN-α receptor (IFNAR) that consists of IFNAR1 and IFNAR2 chains. The type II IFNs only has one member called IFN-γ. Mature IFN-γ is an anti-parallel homodimer, which binds to the IFN-γ receptor (IFNGR) complex to elicit a signal within its target cell. The type III IFN group consists of three IFN-λ molecules called IFN-λ 1, IFN-λ 2 and IFN-λ 3 (also called IL29, IL28A and IL28B respectively). [See Novel interferons, Jan Vilcck, Nature Immunology, 2003, 4, 8-9.] The IFN-λ molecules signal through a receptor complex consisting of IL10R2 (also called CRF2-4) and IFNLR1 (also called CRF2-12). [See Murine interferon lambdas (type III interferons) exhibit potent antiviral activity in vivo in a poxvirus infection model, Bartlett et al., Journal of General Virology, 2005, 86, 1589-1596.]

“Antibody” or “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be humanized or chimeric antibodies. The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques. Antibodies may be for diagnostic purposes or for therapeutic purposes. Examples of therapeutic antibodies include but are not limited to herceptin, rituxan, campath (Mellinium pharma Inc.), gemtuzumab (Cell tech.), herceptin (Genentech), panorex (Centocor GSK), rituximab (Genentech), bexxar (Coraxia GSK), edrecolomab (Glaxo-wellcome), alemtuzumab (ILEX Pharmaceuticals), mylotrag (Whety-Ayerst), IMC-C225, smartin 195, and mitomomab (Imclone systems). Therapeutic antibodies include those coupled to a therapeutic compound and “cold dose” antibodies, such as for reducing non-specific binding. See, e.g., Abrams et al., U.S. Pat. No. RE38,008.

Examples of imaging agents include, but are not limited to, the following: radioisotopes (e.g., 3H, 14C, 35S, 125I, 131I), fluorescent labels (e.g., FITC, rhodamine, lanthanide phosphors), MRI contrast agents (e.g., Gadolinum chelates (Gd)) luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody.

The thioredoxin homologue protein can be conjugated with the active agent by any suitable conjugation strategy. The thioredoxin homologue protein can be conjugated with the active agent by click chemistry. For example, as described below in Example 2, the attachment of vancomycin to C35STRX can be carried out in three stages using click chemistry: 1) conjugation of azido-N-hydroxysuccinimide (NHS) to C35STRX; 2) conjugation of alkyne-PEG ester to vancomycin; and 3) reactive coupling of the linkers. In a preferred embodiment the click chemistry does not utilize copper.

In various embodiments of the invention, an active agent is conjugated to the thioredoxin homologue protein by a linker. The linker can be a cleavable linker or a non-cleavable linker. For example, the conjugation can be achieved through an ester linkage of the thioredoxin homologue protein to the active agent. Thioredoxin homologue proteins of the invention can be conjugated to one or more active agents per molecule. For example, the thioredoxin homologue protein can be conjugated to more than 1 active agent, more than 2 active agents, more than 3 active agents, more than 4 active agents, more than 5 active agents, more than 6 active agents, more than 7 active agents, more than 8 active agents, more than 9 active agents, more than 10 active agents. In these embodiments, the active agents can be the same, different, or some the same and some different. The rate of cleavage of the linker may be controlled by methods known in the state of art such as varying the length or number of moieties such as polyethylene glycol or the sequence or identity of cleavage sites such as ester cleavage sites.

Reactive side chains of the naturally-occurring amino acids lysine (Lys) and cysteine (Cys) are attractive sites of chemical conjugation. In native human thioredoxin, for example, there are twelve Lys on each molecule, allowing for polyvalent conjugation of multiple active agents to a single thioredoxin protein scaffold, but also creating the potential for heterogeneous mixtures. Hence, conjugation conditions are optimized to achieve a consistent average of payload linkage. Means by which payload number (valency) may be manipulated or optimized include but are not limited to controlling the duration of the conjugation reaction or the relative concentrations of reactants. In addition, the number of reactive moieties (such as Lys or Cys residues) present in the thioredoxin homologue protein can be manipulated by genetic means to reduce or increase the number of potential sites for linker attachment.

In preliminary studies, conjugation of linkers to surface Lys residues of monocysteinic active site thioredoxin was found to attenuate its ability to reduce insulin disulfides by 70% vs. unconjugated monocysteinic thioredoxin. Varying the length of the attached linker and increasing the number of PEG did not appreciably improve efficiency. The thioredoxin active site region was blocked by dimerization during conjugation with an average valency of 11 bound linkers (out of 12 possible binding sites) indicating that one Lys was inaccessible to conjugation by the presence of a disulfide-bound target at the thioredoxin active site. Inspection of the crystal structure of monocysteinic thioredoxin bound to an NFkB-derived peptide revealed three Lys in proximity to the binding face, namely Lys positions 72, 94, and 96 of SEQ ID NO: 12. Mutation of one, two or all three of these residues may improve scaffold activity by preventing steric or other adverse interactions resulting from conjugation at these sites. In another embodiment, the present invention includes a monocysteinic thioredoxin analog protein in which amino acid residues at positions corresponding to Lys positions 72, 94, and/or 96 are non-lysine variants, and specifically, one, two or all three can be alanine (Ala) residues. For example, SEQ ID NO:28 illustrates modification of the lysine residues at positions 72, 94, and 96 to be any residue except for lysine and SEQ ID NO: 29 illustrates all three being alanine residues.

NHS esters are a suitable choice for functionalizing amines as are amine functionalized cyclooctyne derivatives such as Dibenzocyclooctyne-amine (DBCO). Cyclooctynes are useful in strain-promoted copper-free azide-alkyne cycloaddition reactions and will react with azide functionalized compounds or biomolecules without the need for a Cu(I) catalyst to result in a stable triazole linkage. Adequate solubility of active agent payloads, such as antibiotics, can be ensured by incorporation of polyethylene glycol (PEG) into the linker structure.

It is important to obtain a uniform reduction state of the final conjugate. To do so, in one embodiment, fully oxidized monocysteinic thioredoxin homolog protein dimers (thus having blocked active site cysteines) are first formed and then used to initiate the conjugation synthesis. Once the conjugation reaction is complete, the disulfide bonds of the dimers are reduced, for example, with DTT. Other suitable reducing agents include, but are not limited to, lipioc acid, NADH or NADPH-dependent thioredoxin reductase, ethylenediaminetetraacetic acid (EDTA), reduced glutathione, dithioglycolic acid, 2-mercaptoehtanol, Tris-(2-carboxyethyl) phoshene, N-acetyl cysteine, NADPH, NADH and other biological or chemical reductants. More specifically, to prepare the linker-conjugated scaffold, oxidized dimers can be first incubated with NHS-PEG (JenKem Technologies USA, Plano, TX) coupled to an TRX azido group. Following conjugation, the dimers can be separated by reducing the disulfide bonds, for example, in 0.1 M DTT in 50 mM Tris (pH 8), incubated for one hour. Reduced azido-thioredoxin homolog conjugates can then be purified, for example, by 5 KDa UF/DF buffer exchange. An active agent is then conjugated by, for example, reacting alkyne-PEG-ester coupled to vancomycin (synthesized by JenKem) under appropriate conditions with the reduced azido-thioredoxin homolog to form a polyvalent thioredoxin homolog-vancomycin conjugate. Unreacted free alkyne-PEG-ester vancomycin can be removed by a final UF/DF step.

In a preferred embodiment using Cu-free click chemistry reactions vancomycin linker: DBCO-PEG-ester is coupled to vancomycin which may be readily reacted with azido groups to facilitate assembly of the final conjugate. In this method Lys residues on monocysteinic Trx are modified to Azido and then these are conjugated to the Vancomycin linker Azido. Fully oxidized monocysteinic thioredoxin homolog protein dimers (thus having blocked active site cysteines) are first formed and then used to initiate the conjugation synthesis. Once the conjugation reaction is complete, the disulfide bonds of the dimers are reduced, for example, with DTT. Other suitable reducing agents include, but are not limited to, lipioc acid, NADH or NADPH-dependent thioredoxin reductase, ethylenediaminetetraacetic acid (EDTA), reduced glutathione, dithioglycolic acid, 2-mercaptoehtanol, Tris-(2-carboxyethyl) phoshene, N-acetyl cysteine, NADPH, NADH and other biological or chemical reductants. Oxidized dimers are first incubated for two hours with NHS-PEG-Azido coupled in a 50 mM HEPES, pH 8 reaction buffer (selected instead of Tris to avoid amines). Following conjugation, Azido-monocysteinic thioredoxin homolog protein conjugates are purified by 5 KDa UF/DF buffer exchange to remove free linker. DBCO-PEG-ester coupled to vancomycin is reacted under appropriate conditions with the Azido-monocysteinic thioredoxin homolog protein to form the polyvalent monocysteinic thioredoxin homolog protein-vancomycin conjugate. Unreacted free DBCO-PEG-ester vancomycin is removed by a UF/DF step. The conjugated material is then reduced with DTT at pH 8. After reduction, the DTT is removed by a UF/DF step.

In one embodiment, a delivery composition of the present invention is used for delivering an active agent to a desired site of action such as an epithelial surface. A composition, including a pharmaceutical composition, can also include, for example, a pharmaceutically acceptable carrier, which includes pharmaceutically acceptable excipients and/or delivery vehicles, for delivering the thioredoxin homologue protein and active agent to a patient. Additionally, a composition, including a pharmaceutical composition of the present invention can be administered to a patient in a pharmaceutically acceptable carrier. As used herein, a pharmaceutically acceptable carrier refers to any substance suitable for delivering the delivery composition useful in the method of the present invention to a suitable in vivo or ex vivo site. Preferred pharmaceutically acceptable carriers are capable of maintaining the delivery composition in a form that, upon arrival of the delivery composition at the desired site, is capable of contacting a mucosal surface. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Preparations for inhalation of therapeutic agents may also include surfactant molecules.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled-release formulation that is capable of slowly releasing a composition of the present invention into a patient. As used herein, a controlled-release formulation comprises one or more therapeutic agents of the present invention in a controlled-release vehicle. Suitable controlled-release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Such controlled-release vehicles may also incorporate reducing agents to maintain an N-terminal monocysteinic active site in a reduced state during storage and delivery.

The optimum amount of delivery composition of the present invention to be administered will vary depending on the route of administration. For instance, if the delivery composition is administered by an inhaled (aerosol) route, the optimum amount to be administered may be different from the optimum amount to be administered by intratracheal microspray or direct topical application. It is important to note that a suitable amount of delivery composition of the present invention is an amount that has the desired function without being toxic to an animal or human. An important benefit of the present invention is that highly effective concentrations of active agents can be administered to a subject without causing toxicity because the active agents can be targeted to specific locations within a subject at mucosal surfaces without the need for systemic administration of the active agent which would require higher doses of active agent, thereby increasing the risk of toxicity. Other routes of administration include but are not limited to oral administration, especially for the treatment of digestive mucus, or topical for the treatment of buccal, nasal, ocular or reproductive mucus.

In a one embodiment of the present invention, a composition, including a pharmaceutical composition, of the present invention that contains a thioredoxin homologue protein having an N-terminal monocysteinic active site conjugated to an active agent is further formulated with one or more agents that maintains the thioredoxin active site in a reduced state following initial reduction using reducing agents. Such reducing agents used in the present invention include, but are not limited to, dithithreitol (DTT), lipioc acid, NADH or NADPH-dependent thioredoxin reductase, ethylenediaminetetraacetic acid (EDTA), reduced glutathione, dithioglycolic acid, 2-mercaptoehtanol, Tris-(2-carboxyethyl) phoshene, N-acetyl cysteine, NADPH, NADH and other biological or chemical reductants.

A delivery composition of the present invention is administered to a patient in a manner effective to deliver the composition, to a target site (e.g., a mucosal surface) at which the activity of the active agent is desired. Suitable administration protocols include any in vivo or ex vivo administration protocol.

According to the present invention, an effective administration protocol (i.e., administering a composition of the present invention in an effective manner) comprises suitable dose parameters and modes of administration that result in contact of the delivery composition with a mucosal surface at or near a location in the body to be treated by the active agent, preferably so that the patient obtains some measurable, observable or perceived benefit from such administration. Effective dose parameters can be determined by experimentation using in vitro samples, in vivo animal models, and eventually, clinical trials if the patient is human. Effective dose parameters can be determined using methods standard in the art for a particular disease or condition. Such methods include, for example, determination of survival rates, side effects (i.e., toxicity) and progression or regression of disease, as well as relevant physiological parameters.

According to the present invention, suitable methods of administering a delivery composition of the present invention to a patient include any route of in vivo administration that is suitable for delivering the composition to the desired site into a patient. The preferred routes of administration will be apparent to those of skill in the art, depending on what part of the body the composition is to be administered, and the disease or condition experienced by the patient. In general, suitable methods of in vivo administration of a delivery composition of the invention include, but are not limited to, dermal delivery, intratracheal administration, inhalation (e.g., aerosol), nasal, oral, pulmonary administration, and impregnation of a catheter. Aural delivery can include ear drops, intranasal delivery can include nose drops or intranasal injection, and intraocular delivery can include eye drops, solid dosage forms, or the use of suitable devices for passage of the drug across the sclera. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can include solids and liquids that can be taken through the mouth, for example, as tablets or capsules, as well as being formulated into food and beverage products or animal feed or feed pellets. Other routes of administration that are useful for mucosal tissues include bronchial, intranasal, other inhalatory, rectal, topical, transdermal, vaginal, transcervical, pericervical and urethral routes. In addition, administration protocols can include pretreatment devices, such as application of the protein, peptide or composition in a diaphragm (e.g., to the cervix) for use in applications such as infertility.

In the methods of the present invention, compositions, including pharmaceutical compositions can be administered to any member of the Vertebrate class, including, without limitation, primates, rodents, livestock, chickens, turkeys, equines and companion animal such as canines and felines. Preferred patients to protect are humans.

A further embodiment of the present invention relates to a method of preventing systemic exposure to an active agent in a patient. The method includes the step of administering the delivery composition to the patient by a delivery route including but not limited to a pulmonary, oral or topical delivery route. The delivery composition can form a covalent bond to its target site once administered and the active agent can act locally at the target site either with or without cleavage. This mechanism of action is distinct from known drug mechanisms of action as many drugs act by molecular interactions wherein ligands bind to receptors with reversible binding of the molecules to the receptors. In yet another preferred embodiment the target site is extracellular and the delivery composition is administered by an extracellular delivery route.

Still another embodiment of the present invention relates to a pharmaceutical composition comprising the delivery composition of the invention and further comprising at least one saccharide or saccharide derivative capable of stabilizing the redox-active thiol group. The saccharide or saccharide derivative can be sucrose, sucralose, lactose, trehalose, maltose, galactose, raffinose, mannose or mannitol. By redox-active thiol group it is meant a thiol group that may exist in either a reduced state (—SH) or an oxidized state (—S—S—). The term “stabilizing” includes, for example, reducing the rate of oxidation of the redox-active thiol group in a reduced state when the polypeptide is present in a pharmaceutical composition with the saccharide or saccharide derivative relative to a composition in which the saccharide or saccharide derivative is omitted. By “saccharide” it is meant any mono-, di-, oligo- or poly-saccharide. Examples of saccharides are glucose, fructose, sucrose, lactose, maltose, galactose, raffinose, inulin, dextran trehalose, sucralose, mannose and mannitol. By saccharide derivative it is meant a compound that structurally resembles the saccharide from which it is derived. For example, sucralose, which is a chlorinated sucrose, would be considered a saccharide derivative of sucrose. Further derivatives include, for example, alditol derivatives for example mannitol and xylitol. Preferred compositions of the present invention comprise non-reducing saccharides, for example raffinose, trehalose, stachyose and particularly sucrose.

Another embodiment of the present invention relates to an animal feed composition comprising the delivery composition of the invention. Examples of animal feed include but are not limited to hay, straw, silage, compressed and pelleted feeds, oils and mixed rations, sprouted grains, legumes, crop residue, grain, cereal crop, and corn. Such feed or feed additive would enable controlled release of conjugated active agents following binding of the monocysteinic thioredoxin homolog protein to the gastrointestinal mucosa.

A further embodiment of the invention, as described in the Examples below, is a delivery composition for sustained epithelial delivery of vancomycin, a powerful glycopeptide anti-infective with bactericidal activity against most gram-positive organisms and bacteriostatic effect on enterococci. Despite concentration-dependent nephrotoxicity, vancomycin is one of the first-line antimicrobials for patients infected with Methicillin-resistant Staphylococcus aureus (MRSA), an independent mortality risk factor associated with rapid lung function decline in cystic fibrosis (CF). Approximately 25% of CF patients have persistent MRSA infection, making them potential candidates for vancomycin treatment. Vancomycin binds to bacterial cell wall precursors and interferes with cell wall synthesis, leading to activation of autolysins and subsequent cell wall destruction. Unlike aminoglycosides used for inhalation (tobramycin, gentamycin, amikacin), vancomycin has significant absorption across epithelia, with inhalation delivery giving nearly 50% of the systemic exposure level as equivalent i.v. Conjugated vancomycin has been investigated as a systemic therapeutic, e.g., for bone targeting, and the inventors of the instant invention have chosen a site for attachment of linkers that has been confirmed in multiple published studies to have no deleterious effect on vancomycin antibacterial activity.

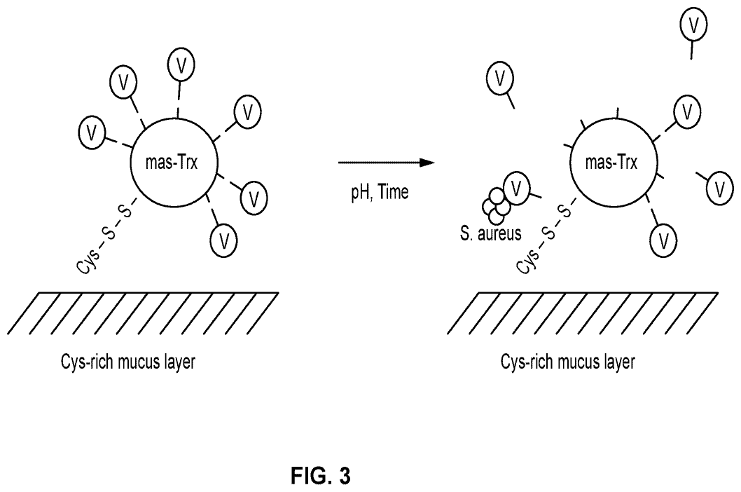

The development of novel thioredoxin homologue protein-antibiotic conjugates utilizing synthetic linkers that are spontaneously cleaved at a slow rate under CF airway pH conditions is described in the examples below. Following inhalation delivery, thioredoxin homologue protein-antibiotic conjugates (synthesized in the fully-reduced form) react with compatible epithelial mucus disulfide bond Cys residues, forming covalent adducts (diagram, left; FIG. 3). The cleavable linker between the thioredoxin homologue protein scaffold and the antibiotic payload subsequently allows for sustained release of free vancomycin from Cys-rich mucus layer via pH-dependent ester cleavage activation over 24 hr (diagram, right; FIG. 3). Even if only soluble and not membrane-associated mucins are targeted, and at the worst-case of fully normal mucus transport rates (20-24 hr mucus clearance), conjugate residence times will ensure significant airway retention of vancomycin as compared to bolus delivery with unrestrained systemic uptake.

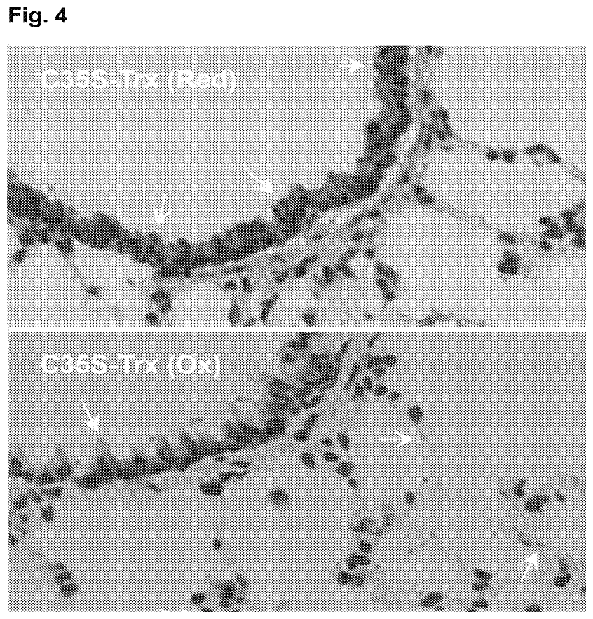

Conversion of the thioredoxin active site to a monothiol allows thioredoxin homologue protein to remain covalently attached to targeted disulfides once it has reacted. A thioredoxin homologue protein having an N-terminal monocysteinic active site forms stable disulfide linkages to target proteins, as verified in vitro by HPLC and by gel-shift, supporting the concept of prolonged thioredoxin homologue protein residence on the airway surface via interaction with both soluble and tethered mucins. Reduced thioredoxin homologue protein can remain bound to extracellular airway mucus for at least 4 hours following intratracheal (IT) delivery to normal rats with normal mucus as visualized by immunohistochemical detection (brown staining; FIG. 4, Top) using anti-human thioredoxin antisera. Inactive (oxidized) thioredoxin homologue protein controls (FIG. 4, Bottom) did not bind mucus or the epithelial surface. While time points later than 4 hours were not included in this study, normalization of mucociliary transport in human airway mucus on epithelia for up to 24 hours is observed confirming similar residence time/mucin binding duration for mucoadhesive conjugates.

No adverse inflammatory effects were observed with high-dose reduced thioredoxin homologue protein in lavage fluid in rat and mice treated by the intratracheal (IT) route of administration and in vitro on human primary bronchial epithelia from CF and normal donors treated apically. No local or systemic toxicity or histopathological anomalies were found in rats dosed acutely with aerosolized drug via nebulizer by the aerosol route of administration at the highest delivered concentration of 40 mg/kg.

A further embodiment of the invention is a composition and method for the treatment of oral cavity cancers. Oral cavity cancer accounts for approximately 3% of all malignancies and is a significant worldwide health problem (Mortazavi, H., et al., 2014, J Dental Res, Dental Clinics, Dental Prospects 8, 6-14). Most oral malignancies occur as squamous cell carcinomas (SCCs); despite advances in treatment modalities, the 5-year survival rate has not significantly improved over the past several decades and still hovers at about 50-60% (up to 75% for mouth floor cancer that has not spread at the time of diagnosis). Many oral SCCs develop from premalignant conditions of the oral cavity. A wide array of conditions have been implicated in the development of potentially premalignant oral epithelial lesions, including leukoplakia, erythroplakia, palatal lesion of reverse smoking, oral lichen planus, oral submucous fibrosis, discoid lupus erythematosus, and hereditary disorders such as dyskeratosis congenital, Franconi anemia and epidermolysis bullosa. Approximately one third of dysplastic lesions and 16% of non-dysplastic progress to carcinoma.

Currently, the treatment of choice for advanced epithelial dysplasia of the oral cavity is surgical excision done by a scalpel, cryosurgery or a CO₂ laser, but such treatment does not usually prevent recurrence. If surgery is not an option, due to the size of the lesion, its location, or the medical status of the patient, available options include observation or chemoprevention with retinoids, epidermal growth factor receptor inhibitors/antagonists, cyclooxygenase-2 inhibitors, p53 modulators, or topical agents such as bleomycin. Antioxidant supplements such as beta-carotene and the retinoids have been the most extensively investigated, especially for leukoplakia, but have not shown promise in the prevention of malignant transformation and recurrence. Thus, there is an unmet medical need for more efficient and long-lasting targeted delivery of drug payloads to premalignant mucosal lesions.

The present invention addresses two factors that influence the effectiveness of drug delivery to the oral cavity. The first is time of retention of the drug delivery system in contact with the oral mucosa; the second is the permeation rate of the drug payload across the oral mucosa. Retaining a drug delivery system in contact with the oral mucosa at a particular location is achieved through the incorporation of mucoadhesive polymers into the formulation. This results in intimate contact with the oral mucosa for a prolonged time, allowing for long duration of drug absorption and a small pathway for diffusion of released drug between delivery system and the mucosal surface. Tethering drugs to the mucosal surface for sustained release over time can extend drug residence at the target site and enhance local bioavailability in epithelial mucus layers. Tuning exposure spatially and temporally improves therapeutic index, enabling safe exposure levels with optimal efficacy. Mucoadhesive interactions have typically been facilitated by the incorporation of thiol groups which form covalent attachment to mucus disulfide bonds. However, chemical thiols as a class have extremely basic pKa (9 to 9.5) and hence are poorly active at the neutral pH of the human oral cavity. To date no mucoadhesive strategy has proven effective for delivery of chemopreventive agents to treat oral premalignancies. Increasing the permeability of the drug through the oral mucosa is another approach used to assure therapeutic levels of a drug via the buccal route, typically through the use of a penetration enhancer in the formulation. Various chemicals have been used as permeation enhancers including surfactants, bile salts, fatty acids and non-surfactants such as cyclodextrins, chitosan and azones. Mucoadhesive polymers and penetration enhancers used for oral mucosal delivery have been extensively reviewed.

In order to overcome the drawbacks of prior approaches, the present invention is a mucus-targeting scaffold based on a monocysteinic active site variant of thioredoxin modified to bind covalently to soluble and membrane-associated mucus proteins and conjugated to a chemotherapeutic agent. A typical epithelial mucin protein has nearly 300 Cys, many in the form of thioredoxin-targetable disulfide bonds. As a monothiol, the thioredoxin homologue protein of the invention lacks the ability to resolve mixed-disulfides and unlike native thioredoxin stays bound covalently to targeted disulfide bonds. This crucially improves its utility as a drug delivery system by prolonging activity via long-duration residence on mucin proteins of epithelial mucus, coupled with attenuated systemic uptake and low toxicity due to sequestration in the mucus layer. Because of its stoichiometric mechanism, the thioredoxin homologue protein can be delivered in a stable, fully-reduced form that does not require additional cofactors. The lack of observed toxic effects in normal animals treated by aerosol at high dose levels suggests that the thioredoxin homologue protein is a safe and effective means of covalent attachment to epithelial mucus in humans.

There are twelve Lys residues on the surface of thioredoxin with primary amines amenable to functionalization and attachment of drug payloads. This embodiment of the invention includes novel monocysteinic thioredoxin-drug conjugates (TDCs) utilizing synthetic linkers that are spontaneously cleaved at a slow rate under physiological pH conditions. Following topical delivery to premalignant lesions on buccal epithelia, TDCs (synthesized in the fully-reduced form) react with compatible epithelial mucus disulfide bond Cys residues, forming covalent adducts, as shown in FIG. 3, left image. The cleavable linker between the monocysteinic thioredoxin scaffold and the drug payload (V) subsequently allows for sustained release of free payload via pH-dependent ester cleavage activation over at least 24 hours, as shown in FIG. 3, right image. Because of the stable covalent bond, conjugate residence times are ample to ensure continuous release of drug to the lesion surface over extended time.

Conversion of the thioredoxin active site to a reduced Cys32 monothiol allows thioredoxin variants to remain covalently attached to targeted disulfides once reacted. Monocysteinic thioredoxin forms stable disulfide linkages to target proteins, as verified in vitro by HPLC and by gel-shift, which supports that monocysteinic thioredoxin has prolonged residence on mucosal surfaces via interaction with both soluble and tethered mucins. In normal rats, reduced monocysteinic thioredoxin remains bound to extracellular airway mucus for at least 4 hours following intratracheal (IT) delivery as visualized by immunohistochemical staining (brown) using anti-human thioredoxin antisera (FIG. 4, top panel). Inactive (oxidized) monocysteinic thioredoxin controls (FIG. 4, bottom panel) did not bind mucus or the epithelial surface. Normalization of mucociliary transport in human airway mucus on epithelia for up to 24 hours has been observed suggesting that residence time of mucoadhesive conjugates is of similar duration.

The following examples are provided for the purpose of illustration and are not intended to limit the scope of the present invention.

### EXAMPLES

### Example 1

To establish the feasibility of covalently binding conjugated monocysteinic active site Cys32-Ser35 thioredoxin (C35STRX) to mucins, conjugation of C35STRX via its N-terminal NH2 to a model payload (biotin) using FZ-Link™ Sulfo-NHS-SS-Biotin (ThermoFisher) was carried out. Dithiothreitol (DTT, 0.1 M) was used to generate active C35STRX conjugates by reduction of the C35STRX active site Cys. Following DTT removal (NAP5 column, GE Healthcare), the mucin binding ability of reduced biotinylated 0.02 C35STRX was evaluated by direct ELISA using 96 well plates 0 (Costar 3370) coated with 100 μl of various concentrations of porcine gastric mucin (Sigma) in 100 mM Tris-HCl buffer (pH 9). 100 μL of biotinylated C35STRX in 50 mM Tris (pH 8) and 1 mM EDTA was then added and incubated for 2 hours, with washes performed between each step. 50 μL of avidin-horseradish peroxidase (HRP) was added and incubated for 30 min followed by 50 μL of tetramethylbenzidine (TMB) substrate. Reactions were stopped after 5 min with 100 μl 1N sulfuric acid. Absorbance at 405 nm (Molecular Devices SpectraMax i3) was plotted against mucin concentration. The observed linear response demonstrated that biotinylated C35STRX conjugates bind mucin in a dose-dependent fashion, confirming that active payloads can be attached to C35STRX without interfering with mucin binding ability.

### Example 2

This example describes the development and characterization of C35STRX-antibiotic conjugates.

### A. Design and Construction of Conjugation Linkers and Antibiotic Payload Conjugates:

C35STRX expression: A codon-optimized synthetic oligonucleotide encoding C35STRX cloned into vector pD861 (Atum, Newark, CA) is expressed in BL21 E. coli under rhamnose induction (4 mM, 37 deg C.) in LB medium with glycerol substrate at an O.D. 600 of 0.8 in 1.5 L shake flasks. Following cell lysis by sonication, protein is purified from clarified extracts using anion exchange chromatography (FPLC, Akta) and 5 kDa ultrafiltration/diafiltration (UF/DF). Purified protein (1 g) reduced with DTT is exchanged into lyophilization buffer prior to an additional UF/DF step under nitrogen to prevent re-oxidation.

Conjugation strategy: The attachment of vancomycin to C35STRX is carried out in three stages using click chemistry: 1) conjugation of azido-N-hydroxysuccinimide (NHS) to C35STRX; 2) conjugation of alkyne-PEG ester to vancomycin; and 3) reactive coupling of the linkers. C35STRX linker: Reactive side chains of the naturally-occurring amino acids lysine and cysteine are attractive sites of chemical conjugation. There are 12 lysines on each C35STRX molecule, allowing for polyvalent conjugation of multiple vancomycin molecules to a single C35STRX scaffold, but also creating the potential for heterogeneous mixtures. Hence, conjugation conditions are optimized to achieve a consistent average of payload linkage. NHS esters were chosen for conjugation as these are the most common choice for functionalizing amines. Adequate solubility of antibiotic payloads is ensured by incorporation of polyethylene glycol (PEG) into the linker structure.

Vancomycin linker: alkyne-PEG-ester is coupled to vancomycin. These are readily reacted with azido groups and facilitate assembly of the final conjugate.

Synthesis and construction of C35STRX-antibiotic conjugates: In order to assure a uniform reduction state of the final conjugate, fully oxidized C35STrx dimers (thus having blocked active site cysteines) are used to initiate the conjugation synthesis prior to reduction with DTT. To prepare the linker-conjugated scaffold, oxidized C35STrx dimers are first incubated for two hours with NHS-PEG-Azido (Quanta Biodesign) coupled in a 50 mM HEPES, pH 8 reaction buffer (selected instead of Tris to avoid amines). Following conjugation, Azido-C35STrx conjugates are purified by 5 KDa UF/DF buffer exchange to remove free linker. DBCO-PEG-ester (Broadpharm) coupled to vancomycin (WuXi STA) is reacted under appropriate conditions with the Azido-C35STRX to form the polyvalent C35STRX-vancomycin conjugate. Unreacted free DBCO-PEG-ester vancomycin is removed by a UF/DF step. The conjugated material is then reduced with DTT at pH 8. After reduction, the DTT is removed by a UF/DF step.

### B. Physical Characterization and Stability Assessment:

C35STRX-antibiotic conjugates are dissolved in PBS to the original volume before lyophilization and assayed for stability over seven days in comparison to non-conjugated reduced C35STRX. C35STRX-antibiotic conjugate concentration is measured by BCA protein assay (Pierce). The percent reduction state of C35STRX-antibiotic conjugates (and hence disulfide-reducing activity) is determined by DTNB (5,5′-Dithiobis-(2-Nitrobenzoic Acid) assay as previously described for TRX (Rancourt, R. C. et al., 2004, Am J Physiol Lung Cell Mol Physiol 286, L931-938). Briefly, 50 μL of 2.5 mM C35STRX-antibiotic conjugate, 175 μL of sample buffer and 25 μL of 6 mM DTNB is incubated in 96-well plates and the absorbance change at 412 nm monitored spectrophotometrically at 30° C. with the reduction state (percentage of free sulfhydryl) determined as actual concentrations of free SH groups divided by theoretical. Degradation/aggregation of C35STRX-antibiotic conjugates is determined by the percentage of C35STRX-antibiotic conjugate monomers assayed by SEC using an Agilent 1100 HPLC system and a BioBasic SEC-300 250×4.6 column (ThermoScientific) with a 40 mM Na acetate, 450 mM Na chloride 2 mM EDTA (pH 5.5) buffer at a flow rate 0.350 mL/min and detection by absorbance at 280 nm (A280).

### C. Determination of Rate and Efficiency of C35STRX-Vancomycin Ester Linker Cleavage at CF and Normal pH:

Ester bonds in the chosen linkers are designed to self-cleave between pH 6 and 8 in a linker and payload-specific manner (Rydholm, A. E. et al., 2007, Acta Biomater 3, 449-455). Release kinetics of the conjugated vancomycin-C35STRX is determined in vitro by pH shift cleavage. C35STRX-antibiotic conjugates stored at pH 5.5 is titrated to pH 6.8 (CF) and 7.6 (normal) and the release rate of vancomycin is measured using a mucin binding assay (described in detail below). Briefly, wells are coated with porcine gastric mucin (10 mg/mL) and then incubated with C35STRX-vancomycin conjugate in PBS. Unbound C35STRX-vancomycin is washed away. At different time points, the solution in each well is replaced with fresh PBS and the released vancomycin quantified using LC/MS.

### D. Evaluation of Alternate Conjugation Conditions (pH, Salt Concentration, Relative Linker/C35STRX Concentrations, Addition of DMSO):

Even though vancomycin is much smaller than the 12 kDa C35STRX scaffold, it is possible that lysine conjugation at certain positions can alter the electrostatic properties (isoelectric points) and hydrophobicity of the C35STRX and result in aggregation, which could influence conjugate stability. This is assessed via the functional assays described below and lysine conjugation conditions adjusted if necessary. Modified versions of the vancomycin linker structure are synthesized in order to accelerate or attenuate pH-dependent ester cleavage.

### Example 3

This example describes the in vitro functional characterization of cleavable C35STRX-vancomycin.

### A. Determination of Payload Valency of Conjugated C35STRX-Vancomycin:

The payload valency of C35STRX-vancomycin under different conjugation conditions is determined using electrospray ionization LC/MS, similar to DAR (drug to antibody ratio) characterization of antibody-drug conjugates (Basa, L., 2013, Antibody-Drug Conjugates, L. Ducry, ed., pp. 285-293, Humana Press, Totowa, NJ). C35STRX-vancomycin conjugate samples are loaded onto a RP column and eluted using an acetonitrile gradient. The desalted C35STRX-antibiotic conjugate fraction is analyzed by MS and the integrated mass peak areas are used to determine average payload (number of antibiotic molecules divided by the number of C35STRX). Payload valencies are compared to in vitro activity in order to optimize the drug: C35STRX ratio. Removal of any contaminating free C35STRX is confirmed by SEC-HPLC and SDS-PAGE.

### B. Determination of In Vitro Activity of C35STRX-Vancomycin Scaffold:

Mucin binding assay: 96 well plates (Costar 3370) are coated with 100 μL of porcine gastric mucin at 10 mg/mL in 100 mM Tris-HCl buffer (pH 9) and incubated overnight at 4° C., washed 3× with PBS/0.05% Tween, then incubated 2 hours with 100 μL of C35STRX-vancomycin in 50 mM Tris (pH 8) and 1 mM EDTA. Biotinylated C35STRX is used as the positive control. Wells are washed 3× and incubated with biotinylated anti-human TRX1 antibody (Abfrontier LF-EK0125) for one hour, washed 3× and incubated with Avidin-HRP for 30 minutes. Samples are washed 3× and incubated with TMB substrate until color change. Reactions are stopped with 100 μL 1M sulfuric acid and A450 nm of the C35STRX-antibiotic conjugates are plotted against biotinylated C35STRX to determine relative mucin binding activity.

Insulin reduction assay: A functional assay is utilized to determine if the enzymatic activity of the C35STRX scaffold is affected differentially by varying vancomycin payload valency, reflecting the potential effect of conjugation to different surface-exposed lysines. RP-HPLC is used to monitor changes over time in the large dimer peak of heterodimeric insulin due to C35STRX reduction activity. The relative activity of different valencies of C35STRX-vancomycin or C35STRX alone is compared over 60 min following incubation with 10 mg/mL insulin. Reactions are stopped by addition of thiolyte and trifluoroacetic acid (TFA) and RP-HPLC (Agilent 1100) is used to quantify the rate of change in area under the insulin dimer peak.

C. Evaluation of Anti-Bacterial Activity Vs. Staphylococcus aureus of Vancomycin+Linker Payload

MIC assay: Anti-bacterial activity of vancomycin+linker cleaved at the ester bond to mimic release from C35STRX conjugates is compared to commercial vancomycin. Based on published data (e.g., Sheikh, S. et al., 2012, Med Chem 8, 1163-1170; Yarlagadda, V., et al., 2015, J Antibiot (Tokyo) 68, 302-312; Mishra, N. M. et al., 2015, Org Biomol Chem 13, 7477-7486) the vancomycin+linker is at least as active as free vancomycin. S. aureus (ATCC 25923, Manassas, VA) is maintained aerobically on trypticase soy agar (TSA) plates at 37° C. Bacteria picked from single colonies is cultured aerobically in Mueller-Hinton broth (MHB) at 37° C. for 24 hours, and then suspended in sterile saline at a density equivalent to that of the 0.5 McFarland standard for spectrophotometry (optical density at 600 nm=1.0). Bacterial suspensions with a concentration of 105 cfu/mL is used to determine minimum inhibitory concentrations (MIC) using standard broth microdilution methods. Vancomycin (Sigma) is used as the positive control and saline is used as negative control. Briefly, two-fold serial dilutions of free vancomycin+linker or controls are added to the wells of sterile 96-well plates containing inoculated NB medium (100 μL) with bacterial cells (105 cfu/mL) at final concentrations ranging from 15.63 to 2,000 μg/mL. MIC is determined as the lowest concentration completely inhibiting bacterial growth over 24 hours at 37° C.

### D. Determination of Bioactivity of Cleavable C35STRX-Antibiotic Conjugate Scaffold and Payload:

Potential loss of C35STRX activity at high valencies: active biotinylated C35STRX were produced which maintained full mucin binding activity with linear kinetics. Conjugating vancomycin to certain lysine positions or at certain payload valency may differentially interfere with C35STRX disulfide recognition or target reduction, as the inventors have observed that occupancy of an average of 11 Lys primary amines by NHS linkers in unoptimized preliminary studies attenuated ⅔ of the disulfide reducing activity of unconjugated C35STRX. While ⅓ of full activity is likely to be more than sufficient to occupy available TRX-targetable mucus disulfides, optimizing lysine amine conjugation conditions should nonetheless be feasible as Lys close to the active site will be less accessible to conjugation than surface-exposed residues and hence easiest to avoid by titration. Full active site occlusion is unlikely as this region is blocked by dimerization during conjugation.

In vitro mucin binding: Porcine mucin is commonly used in binding assays instead of human mucus or sputum due to its uniformity and ready commercial availability. In contrast, human CF patient sputum is particularly difficult to utilize for quantitative binding assays as it is physically heterogeneous and has a highly variable mucin content. The inventors have verified that porcine mucin solutions at the concentrations being used in the quantitative binding assay contain numerous TRX-targetable disulfide bonds, and are functionally similar to human mucus with respect to the dose-dependency of TRX or C35STRX activity.

### Example 4

This example evaluates the pharmacokinetics, lung inflammation and airway residence time in mice of intratracheally administered sustained-release C35STRX-vancomycin vs. free vancomycin+linker.

Six-week-old female BALB/c mice (six per group) are administered a single IT dose (1 mg/kg) of 1) free linker-conjugated vancomycin, or 2) C35STRX-vancomycin conjugate in either the reduced (active) or 3) oxidized (inactive control) form. The PK profile of free vancomycin+linker (IT) is compared to that of a fourth group dosed i.v. bolus with vancomycin+linker (1 mg/kg). At five time points post-dose (0.5, 1, 2, 6 and 24 hours), 0.05 mL blood samples (n=3 mice for 0, 1, 6 hour and n=3 mice for 0.5, 2, 24 hour) are collected for separation of serum and determination of vancomycin PK profiles including Cmax, Tmax, AUC and T1/2. A total of 18 mice (n=6 mice per three groups) are treated via the IT route and six mice are treated via i.v. (24 animals total, with sacrifice at final time point). Vancomycin+linker concentrations are determined using LC/MS and PK parameters are calculated from the concentration-time profile using Kinetica (AlfaSoft). For lung inflammation and airway residence time evaluation, at two time points post-dose (6 and 24 hours), lung tissues are harvested with left lobes prepared for histological hematoxylin and eosin staining (H&E) and right lobes for immunohistochemistry in order to quantify bound vancomycin using an anti-vancomycin antibody (Abcam, ab15075). The degree of peribronchial and perivascular inflammation is evaluated from H&E based on a 5-point quantitative scoring system described by Duan et al., 2008, J Immunol 181, 8650-8659. The distribution of vancomycin (frequency, intensity and location of staining) is quantitatively evaluated with Provantis® Pathology software (Alizee Pathology, Maryland) in order to evaluate relative residence times and clearance.

### Example 5

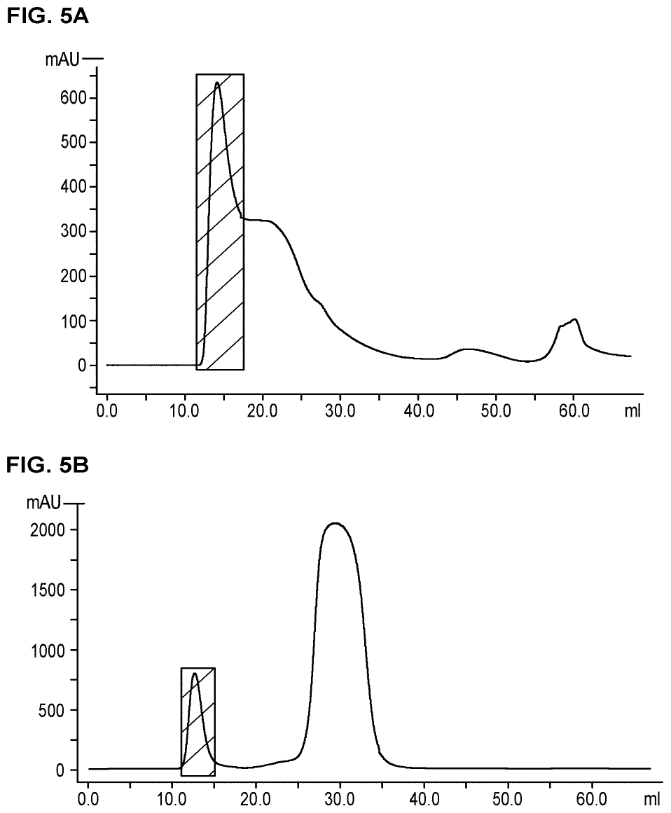

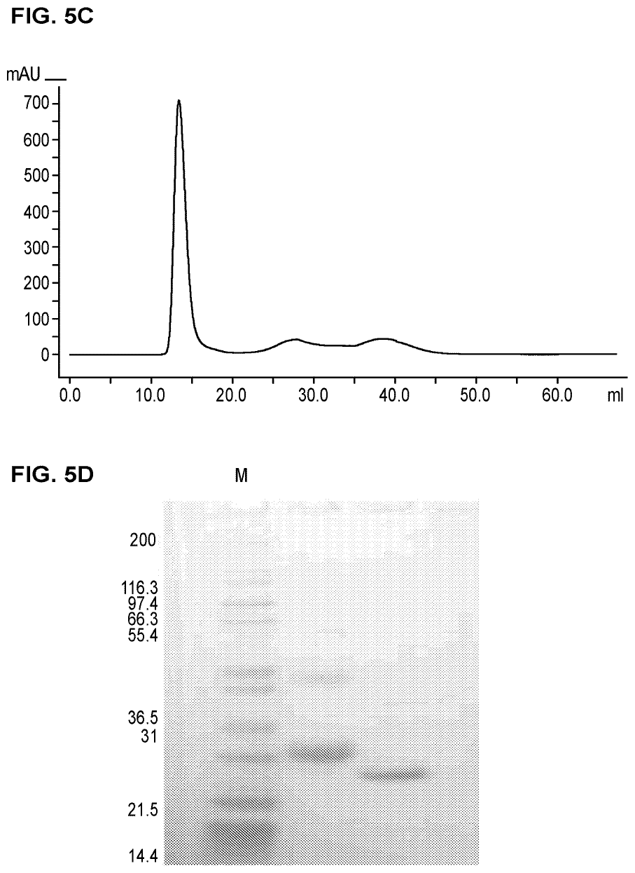

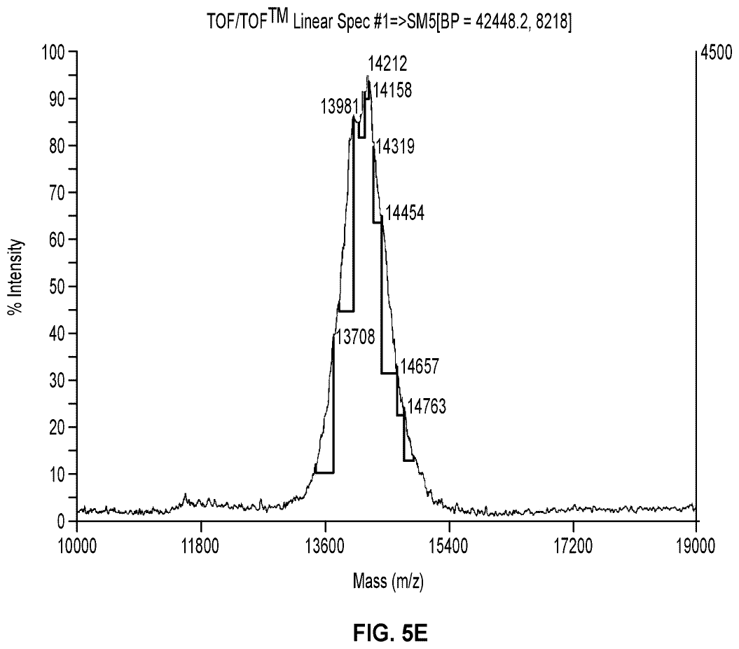

This example demonstrates that the inventors were able to conjugate up to 11 linkers to C35STRX lysines without eliminating the ability of the C35STRX to reduce target protein disulfides. C35STRX was oxidized and Lys residues were conjugated to an azido-PEG linker. The conjugated material was then reduced and tested for molecular weight using SDS-PAGE (FIG. 5D) and MALDI-TOF (FIG. 5E) and activity assessed using a single-turnover insulin protein disulfide reduction assay. From the gel and MALDI-TOF results there is no free C35STRX; however, the conjugated material retained about 33% of its activity in the insulin assay as compared to unreacted reduced C35STRX. See FIGS. 5A-5E and their descriptions above.

TABLE 1
DTNB Assay:

| | A₂₈₀ nm | | A₄₁₂ nm | | |
|---|---|---|---|---|---|
| | OD | mM | OD | mM | % reduced |
| C35STRX | 11.8 | 1.69 | 1.22 | 1.85 | 109.4 |
| Azido-C35STRX | 4.0 | 0.57 | 0.40 | 0.61 | 106.6 |

Insulin Activity Assay with C35STRX and Azido-C35STRX:
C35STRX OD₂₈₀ was 11.8, corresponding to a concentration of 1.69 mM.
C35STRX a OD₂₈₀ was 15, corresponding to a concentration of 2.14 mM.
Azido-C35STRX OD₂₈₀ was 4.0, corresponding to a concentration of 0.57 mM.
pH 8 Insulin stock: 143 mL of Tris pH8 and 572 □L H₂O and 25 □L of insulin (10 mg/mL) and 10 □L of 0.5 M EDTA.
C35STRX stock was 0.5 mM.
Azido-C35STRX stock was 0.5 mM concentration.

TABLE 2

| | | | Azido- | | pH 8 Insulin |
|---|---|---|---|---|---|
| | C35STRX | | C35STRX | | stock |
| C35STRX time 0 | 50 | □L | 0 | □L | 150 □L |
| C35STRX time 90 | 50 | □L | 0 | □L | 150 □L |
| Azido- C35STRX time 0 | 0 | □L | 50 | □L | 150 □L |
| Azido- C35STRX time 90 | 0 | □L | 50 | □L | 150 □L |
| Insulin | 0 | □L | 0 | □L | 150 □L |

The reaction was stopped by addition of 10 □L of 1 Miodoacetic acid and 400 □L of 0.1% TFA. Samples were analyzed by RP-HPLC for changes in the insulin heterodimer peak.

TABLE 3

| | | % reduction | Azido- | % reduction Azido- |
|---|---|---|---|---|
| Time | C35STRX | C35STRX | C35STRX | C35STRX |
| 0 min | 8515.4 | 0 | 8959.8 | 0 |
| 90 min | 50.2 | 99.40 | 6030.6 | 32.69 |

TABLE 4

| | | % reduction | Azido- | % reduction Azido- |
|---|---|---|---|---|
| Time | C35STRX | C35STRX | C35STRX | C35STRX |
| 0 min | 10602 | 0 | 10213 | 0 |
| 90 min | 243.2 | 97.70 | 6835 | 33.07 |

### Example 6

This Example demonstrates improvements in the in vitro catalytic activity and target-binding efficiencies of monocysteinic thioredoxin analog variants having Lys bound linkers by mutation of Lys residues closest to the binding pocket of the thioredoxin analog.

In preliminary studies, conjugation of linkers to surface Lys residues of monocysteinic thioredoxin was found to attenuate its ability to reduce insulin disulfides by 70% vs. unconjugated monocysteinic thioredoxin. Varying the length of the attached linker and increasing the number of PEG did not appreciably improve efficiency. The thioredoxin active site region was blocked by dimerization during conjugation with an average valency of 11 bound linkers (out of 12 possible binding sites) indicating that one Lys was inaccessible to conjugation by the presence of a disulfide-bound target at the thioredoxin active site. Inspection of the crystal structure of monocysteinic thioredoxin bound to an NFkB-derived peptide revealed three Lys in proximity to the binding face. Mutation of one or more of these residues therefore improves scaffold activity by preventing steric or other adverse interactions resulting from conjugation at these sites. Ala mutations at Lys positions 72, 94 and 96 are constructed and evaluated for insulin reduction activity both before and after conjugation to non-cleavable NHS linkers attached to an IR-dye marker molecule.

### Construction and Characterization of Optimized Lys Variant C35S(KA)TRX Binding Scaffolds

Codon-optimized synthetic oligonucleotides encoding Lys to Ala mutants of C35STRX cloned into vector pD861 (Atum, Newark, CA) are expressed soluble in BL21 ArhaB E. coli under rhamnose induction (0.1% w/v, 26 deg C.) in APF-LB medium with 0.05% w/v glucose substrate at an O.D.₆₀₀ of 0.8 in 2.8 L Fernbach shake flasks at NRC (Montreal Canada). Following cell pellet lysis (EmulsiFlex-C3, Avestin, Ottawa Canada) protein is purified from clarified extracts using anion exchange chromatography (FPLC, Akta) and 5 kDa ultrafiltration/diafiltration (UF/DF). Purified C35S(KA)TRX proteins (1 g) reduced with DTT are exchanged into lyophilization buffer prior to an additional UF/DF step under nitrogen to prevent re-oxidation. Physical characterization: C35S(KA)TRX concentration is measured by BCA Protein assay (Pierce). The percent reduction state (and hence potential disulfide-reducing activity) is determined by DTNB (5,5′-Dithiobis-(2-Nitrobenzoic Acid) assay. Briefly, 50 μl of 2.5 mM C35S(KA)TRX, 175 μl of sample buffer and 2.5 μl of 6 mM DTNB is incubated in 96-well plates and the absorbance change at 412 nm monitored spectrophotometrically at 30° C. with the reduction state (percentage of free sulfhydryl) calculated by actual concentrations of free SH groups divided by theoretical.

Degradation/aggregation state is determined by the percentage of C35S(KA)TRX monomers assayed by SEC using an Agilent 1100 HPLC system and a BioBasic SEC-300 250×4.6 column (ThermoScientific) with a 40 mM Na acetate, 450 mM Na chloride 2 mM EDTA (pH 5.5) buffer at a flow rate 0.350 mL/min and detection by absorbance at 280 nm (A280).

Functional characterization: C35S(KA)TRX variant activity vs. C35STRX is assayed using a modified insulin disulfide reduction assay based on RP-HPLC to monitor changes over time in the large dimer peak of heterodimeric insulin due to protein disulfide reduction. The relative activities of the three Lys mutants vs. C35STRX are determined over 60 min following incubation with 0.25 mg/ml. insulin. Reactions are stopped by addition of iodoacetic acid and trifluoroacetic acid (TFA) and RP-HPLC (Agilent 1100) is used to quantify the rate of change in area under the insulin dimer peak.

### Characterization of Lys Variant C35S(KA)TRX Binding Scaffolds Conjugated to IRDye750

Following analysis of the three Lys variants of C35STRX to confirm that Lys mutation does not markedly impact structure and function, their insulin-reduction and target-binding activities vs. C35STRX are then evaluated after conjugation to the remaining exposed Lys. For this evaluation, a readily quantifiable fluorescent dye coupled to a non-cleavable N-hydroxysuccinimide (NHS) ester reactive group that provides functionality for labeling primary amines of Lys is used. IRDye® 750 NHS Ester (LiCor, Lincoln NE) is an infrared dye with detection near 750 nm. In order to assure a uniform reduction state of the dye conjugates, fully oxidized dimers with blocked active site cysteines of 1) Lys variants of C35S(KA)TRX and 2) control C35STRX are used as the substrates for conjugation prior to reduction with DTT. Dimers of each protein scaffold (verified by SDS-PAGE and SEC-HPLC) are incubated for two hours with IRDye® 750 NHIS Ester in a 50 mM HEPES, pH 8 reaction buffer, selected instead of Tris to avoid amines. Following conjugation, 0.1 M DTT is added in 50 mM Tris pH 8 and incubated one hr. Residual DTT is purified away from the reduced dye-scaffold conjugates by 5 KDa UF/DF buffer exchange.

Characterization: 1. Residual insulin reduction activity is analyzed by RP-HPLC as for the unconjugated scaffolds. 2. Relative substrate affinity K_{D} and association rate kₒₙ for binding to insulin disulfides (chosen over mucin for simplicity and better signal to noise) are quantified in vitro using surface plasmon resonance (OpenSPR, Nicoya LifeSciences, Kitchener, Ontario Canada). The best-performing conjugated Lys variant scaffold that retain insulin disulfide activity greater than original conjugated C35STRX, or exhibits improved affinity/binding kinetics, is used as the final CSS(KA)TRX delivery scaffold for further studies. This final optimized scaffold conjugated to IRDye750 is further characterized for identity and MW by LC/MS, purity by SDS-PAGE, and SEC-HPLC for aggregation state and linker valency.

### Interpretation of Results/Anticipated Results

Efficient binding of dye-conjugated C35STRX to mucin and retention of significant insulin reduction activity vs. unconjugated C35STRX will demonstrate that there is in vitro feasibility for the use of monocysteinic thioredoxin to target mucosal surfaces for controlled drug payload delivery with ≥30% of the disulfide-reduction activity of non-conjugated C35STRX.

### Example 7

This example investigates the kinetics and selectivity of epithelial target binding in vivo by conjugation of monocysteinic thioredoxin analog variants having lysine linkers bound to a non-cleavable infra-red fluorescent dye with topical delivery to the oral epithelium of rodents followed by oral and whole-body in vivo imaging to visualize tissue distribution and concentration.

The central obstacle for development of therapeutic strategies to target buccal premalignant lesions has been poor selective binding to mucosal surfaces. Such binding is essential to establish the sustained, high local drug concentrations needed to eliminate premalignant cells. In order to evaluate the suitability of C35STRX as a candidate delivery technology, in vivo imaging of IR-dye conjugated C35STRX applied topically to buccal membranes in mice is used.

### In Vivo Imaging of Tissue Distribution and Concentration of Topically Applied IRDye750-C35STRX Conjugate in Mice

The final optimized scaffold candidate identified in Example 6 above is used to deliver IRDye750 to the buccal membranes of anaesthetized mice following which an imaging time course is conducted. As a control for dye visualization, unconjugated IRDye® 750 is used and as a negative control for binding IR dye-conjugated (oxidized) C35STRX scaffold that lacks target reactivity is used. For each treatment or control, six-week-old female BALB/c mice (six per group) are used. Animals are anesthetized under IRB-approved protocols and treated topically by application of microliter volumes of increasing concentrations of reduced dye conjugates or controls following which they are imaged over a 6 hour time course starting at one hour post-exposure. Images are captured digitally and analyzed for dye conjugate concentration (by intensity) and distribution. Saphenous vein blood samples (50 μl) are taken at six hr for assessment of albumin binding.

### Interpretation of Results/Anticipated Results

These experiments quantify the ratio of dye present at the original oral epithelial application site vs. dye distributed gastrically (swallowed) or systemically (taken up by epithelial cells). Differences between reduced and oxidized dye-scaffold conjugates allow a determination of the binding kinetics over a time course and an assessment of drug-delivery feasibility using this approach.

### Example 8

This example quantifies cleavage and release of a drug payload conjugated to the optimized monocysteinic thioredoxin scaffold via a range of linkers containing cleavable ester bonds and evaluates the ability of the released drug to penetrate mucosal cells by treatment of buccal epithelium ex vivo followed by immunohistochemical assessment of drug activity in tissue sections stained for Annexin-V, a marker of chemotherapy-induced apoptosis.

The in vitro cleavage rate from the C35STRX scaffold of an auristatin (monomethyl auristatin E-MMAE) drug payload bound to thioredoxin substrate in vitro with detection by ELISA is first assessed. The selection of MMAE as the cytotoxin is based on its extensive use in approved ADCs, the availability of high quality anti-MMAE antibodies for imaging, and the commercial availability of various forms of linker-MMAE. Several different linkers are utilized in order to select a design with optimal release kinetics based on the unique physical interactions of the payload and C35STRX scaffold. Mucosal permeability of the best-performing linker-scaffold conjugate is then be evaluated ex vivo following topical application onto sheep buccal mucosa by sectioning and immunohistochemical staining for drug using anti-MMAE and apoptosis using an anti-Annexin V antibody.

### Construction and In Vitro/Ex Vivo Characterization of MMAE-C35STRX Drug Conjugate

Two to four NHS-PEG and DBCO-PEG linkers with variable length cleavable ester bonds and PEG repeats coupled to MMAE are synthesized at 1 g scale at WuXi STA. These are conjugated to C35STRX and characterized physically and functionally as described above for dye conjugates. The rate of release for each conjugate is determined using an anti-MMAE ELISA detection time course following reaction of reduced conjugates with an immobilized insulin disulfide substrate. The MMAE linker conjugate is further characterized for drug penetration into buccal mucosa from sheep obtained from a local abattoir and used within 2 h of slaughter. For each assay, the buccal epithelium is separated from underlying connective tissues with surgical scissors and layered onto a hydrogel supplied by medium from below. Various concentrations of MMAE-C35STRX conjugate in 2.5 μl volumes are applied to the mucosal surface and incubated for various times (30 min to 6 hr) then washed to remove unabsorbed drug. The treated sections are excised individually and fixed for sectioning and IHC (at Alizee) with antisera to human thioredoxin, MMAE and Annexin-5. Controls are unconjugated MMAE and reduced C35STRX.

### Interpretation of Results/Anticipated Results

The ability of MMAE released over time from bound C35STRX to penetrate mucosal epithelium to at least three cell layers by MMAE is anticipated to be demonstrated, confirming utility for treatment of premalignant lesions.

Each of the publications and other references discussed or cited herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

### REFERENCES

1. Dunnhaupt, S., Kammona, O., Waldner, C., Kiparissides, C. & Bernkop-Schnurch, A. Nano-carrier systems: Strategies to overcome the mucus gel barrier. Eur J Pharm Biopharm 96, 447-453 (2015).
2. Jordan, C., Shuvaev, V. V., Bailey, M., Muzykantov, V. R. & Dziubla, T. D. The Role of Carrier Geometry in Overcoming Biological Barriers to Drug Delivery. Curr Pharm Des 22, 1259-1273 (2016).
3. Cone, R. A. Barrier properties of mucus. Adv Drug Deliv Rev 61, 75-85 (2009).
4. Chmiel, J. F. et al. Antibiotic management of lung infections in cystic fibrosis. I. The microbiome, methicillin-resistant Staphylococcus aureus, gram-negative bacteria, and multiple infections. Ann Am Thorac Soc 11, 1120-1129 (2014).
5. Biller, J. A. Inhaled antibiotics: the new era of personalized medicine? Curr Opin Pulm Med 21, 596-601 (2015).
6. Quon, B. S., Goss, C. H. & Ramsey, B. W. Inhaled antibiotics for lower airway infections. Ann Am Thorac Soc 11, 425-434 (2014).
7. Netsomboon, K. & Bernkop-Schnurch, A. Mucoadhesive vs. mucopenetrating particulate drug delivery. Eur J Pharm Biopharm 98, 76-89 (2016).
8 Shaikh, R., Raj Singh, T. R., Garland, M. J., Woolfson, A. D. & Donnelly, R. F. Mucoadhesive drug delivery systems. J Pharm Bioallied Sci 3, 89-100 (2011).
9. Salama, R., Traini, D., Chan, H.-K. & Young, P. M. Recent Advances in Controlled Release Pulmonary Therapy. Current Drug Delivery 6, 404-414 (2009).
10. Ijaz, M. & Bernkop-Schnurch, A. Preactivated thiomers: their role in drug delivery. Expert Opin Drug Deliv 12, 1269-1281 (2015).
11. Holmgren, A. Thioredoxin. Annu Rev Biochem 54, 237-271 (1985).
12. Yashiro, M. et al. Redox-active protein thioredoxin-1 administration ameliorates influenza A virus (H1N1)-induced acute lung injury in mice. Critical care medicine 41, 171-181 (2013).
13. Han, G.-Y., Lee, E.-K., Park, H.-w., Kim, H.-J. & Kim, C.-W. Exogenous rhTRX reduces lipid accumulation under LPS-induced inflammation. Exp Mol Med 46, e71 (2014).
14. Sato, A. et al. Thioredoxin-1 Ameliorates Cigarette Smoke-Induced Lung Inflammation and Emphysema in Mice. Journal of Pharmacology and Experimental Therapeutics 325, 380-388 (2008).
15. Torii, M. et al. Thioredoxin suppresses airway inflammation independently of systemic Th1/Th2 immune modulation. European Journal of Immunology 40, 787-796 (2010).
16. Jaeger, S. U. et al. Cell-mediated reduction of human B-defensin 1: a major role for mucosal thioredoxin. Mucosal immunology 6, 1179-1190 (2013).
17. Schroeder, B. O. et al. Reduction of disulphide bonds unmasks potent antimicrobial activity of human B-defensin 1. Nature 469, 419-423 (2011).
18. Du, Y., Zhang, H., Lu, J. & Holmgren, A. Glutathione and Glutaredoxin Act as a Backup of Human Thioredoxin Reductase 1 to Reduce Thioredoxin 1 Preventing Cell Death by Aurothioglucose. Journal of Biological Chemistry 287, 38210-38219 (2012).
19. Holmgren, A. Thioredoxin and glutaredoxin systems. J Biol Chem 264, 13963-13966 (1989).
20. Das, K. C. & White, C. W. Redox systems of the cell: possible links and implications. Proc Natl Acad Sci USA 99, 9617-9618 (2002).
21. Hanschmann, E. M., Godoy, J. R., Berndt, C., Hudemann, C. & Lillig, C. H. Thioredoxins, glutaredoxins, and peroxiredoxins—molecular mechanisms and health significance: from cofactors to antioxidants to redox signaling. Antioxid Redox Signal 19, 1539-1605 (2013).
22. Rubartelli, A., Bajetto, A., Allavena, G., Wollman, E. & Sitia, R. Secretion of thioredoxin by normal and neoplastic cells through a leaderless secretory pathway. J Biol Chem 267, 24161-24164 (1992).
23. Joo, N. S. et al. Proteomic analysis of pure human airway gland mucus reveals a large component of protective proteins. PLOS One 10, e0116756 (2015). 24. Yuan, S. et al. Oxidation increases mucin polymer cross-links to stiffen airway mucus gels. Science Translational Medicine 7, 276ra227 (2015).
25. Roos, G. et al. How Thioredoxin Dissociates Its Mixed Disulfide. PLOS Comput Biol 5, e1000461 (2009).
26 Rancourt, R. C., Lee, R. L., O'Neill, H., Accurso, F. J. & White, C. W. Reduced thioredoxin increases proinflammatory cytokines and neutrophil influx in rat airways: modulation by airway mucus. Free Radic Biol Med 42, 1441-1453 (2007).
27 Rancourt, R. C. et al. Thioredoxin liquefies and decreases the viscoelasticity of cystic fibrosis sputum. Am J Physiol Lung Cell Mol Physiol 286, L931-938 (2004).
28 Heifetz, P. B., Moskowitz, H., Nichols, D. & Rancourt, R. C. pH dependency of mucolytic agents: the case for protein-drug mucus normalization. Pediatric Pulmonology 50, S235 (2015).
29. Heifetz, P. B., Rancourt, R. C. & Nichols, D. A novel inhaled mucus normalizing therapy for cystic fibrosis. Pediatric Pulmonology 49, S307 (2014).
30. Falk Libby, E. et al. Theradux™, a novel biologic derived from optimized Thioredoxin-1, improves mucociliary transport in situ. Pediatric Pulmonology 51, S292-293 (2016).
31. Bamgbola, O. Review of vancomycin-induced renal toxicity: an update. Therapeutic Advances in Endocrinology and Metabolism 7, 136-147 (2016).
32. Navarre, W. W. & Schneewind, O. Surface proteins of gram-positive bacteria and mechanisms of their targeting to the cell wall envelope. Microbiol Mol Biol Rev 63, 174-229 (1999).
33. Sullivan, B. P., El-Gendy, N., Kuehl, C. & Berkland, C. Pulmonary Delivery of Vancomycin Dry Powder Aerosol to Intubated Rabbits. Molecular Pharmaceutics 12, 2665-2674 (2015). 34 Albayati, Z. A. F. et al. Novel Bone-Targeting Agent for Enhanced Delivery of Vancomycin to Bone. Antimicrobial Agents and Chemotherapy 60, 1865-1868 (2016).
35. Sheikh, S. et al. Evaluating the diagnostic and chemotherapeutic potential of vancomycin-derived imaging conjugates. Med Chem 8, 1163-1170 (2012). 36 Arnusch, C. J., Pieters, R. J. & Breukink, E. Enhanced membrane pore formation through high-affinity targeted antimicrobial peptides. PLOS One 7, e39768 (2012).
37. Yarlagadda, V., Konai, M. M., Manjunath, G. B., Ghosh, C. & Haldar, J. Tackling vancomycin-resistant bacteria with ‘lipophilic-vancomycin-carbohydrate conjugates’. J Antibiot (Tokyo) 68, 302-312 (2015).
38. Mishra, N. M. et al. Evaluation of the antibacterial and antibiofilm activities of novel CRAMP-vancomycin conjugates with diverse linkers. Org Biomol Chem 13, 7477-7486 (2015).
39. Meng, X. et al. Sensitive Detection of Staphylococcus aureus with Vancomycin-Conjugated Magnetic Beads as Enrichment Carriers Combined with Flow Cytometry. ACS Applied Materials & Interfaces 9, 21464-21472 (2017).
40. Rydholm, A. E., Anseth, K. S. & Bowman, C. N. Effects of neighboring sulfides and pH on ester hydrolysis in thiol-acrylate photopolymers. Acta Biomater 3, 449-455 (2007).
41. Basa, L. in Antibody-Drug Conjugates. (cd. L. Ducry) 285-293 (Humana Press, Totowa, NJ; 2013).
42. Duan, W., So, T. & Croft, M. Antagonism of airway tolerance by endotoxin/lipopolysaccharide through promoting OX40 L. and suppressing antigen-specific Foxp3+T regulatory cells. J Immunol 181, 8650-8659 (2008).

1-42. (canceled)

43. A method for sustained release of an active agent to an epithelial surface in the body, comprising administering to a subject a delivery composition comprising
(i) a thioredoxin homolog protein having an N-terminal monocysteinic active site, wherein the cysteine residue of the active site is in a reduced state; and
(ii) an active agent conjugated to the thioredoxin homolog protein;
wherein the delivery composition is prepared by first conjugating the thioredoxin homolog protein having an N-terminal monocysteinic active site to the active agent and then reducing the cysteine residue of the active site, wherein the active agent is conjugated to the thioredoxin homolog protein by a cleavable linker

44. The method of claim 43, wherein the cleavable linker is a cleavable ester linker.

45. The method of claim 43, wherein more than one active agent is conjugated to the thioredoxin homolog protein, and wherein the active agents comprise the same or different molecular entities.

47. The method of claim 43, wherein the thioredoxin homolog protein has an active site in which C-terminal active site cysteine is replaced with serine.

48. The method of claim 43, wherein the linker is attached to the thioredoxin homolog protein at a lysine residue.

49. The method of claim 43, wherein the thioredoxin homolog protein comprises more than five linkers.

50. The method of claim 43, wherein more than five active agents are conjugated to the thioredoxin homolog protein, and wherein the active agents are the same or different.

51. The method of claim 43, wherein the active agent is selected from the group consisting of a therapeutic active agent, a diagnostic active agent, and an imaging active agent.

52. The method of claim 43, wherein the active agent is a therapeutic active agent.

53. The method of claim 52, the therapeutic active agent is selected from the group consisting of anti-infectives, radionuclides, chemotherapeutic agents; and cytotoxic agents.

54. The method of claim 52, wherein the therapeutic active agent is an anti-infective selected from the group consisting of vancomycin, tobramycin, amikacin, ciprofloxacin, levofloxacin, colistin, aztreonam, gentamicin, polymyxin B, fosfomycin, ceftazidime, meropenem, carbopenem, imipenem, cefepime, and piperacillin.

55. The method of claim 52, wherein the therapeutic active agent is an chemotherapeutic agent selected from the group consisting of monomethyl auristatin E (MMAE), methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamoxifen, paclitaxel, vincristine, vinblastine, camptothecin, actinomycin D, cytarabine, combrestatin, cyclosporine A, or lifitegrast.

56. The method of claim 43, wherein the delivery composition is formulated for delivery by a route selected from the group consisting of oral, topical and inhalation.


SEQUENCE LISTING (29 sequences)

SEQ ID NO: 1 - protein, 4 aa, synthetic construct
  REGION 1..4: note = synthetic peptide motif
CGPC

SEQ ID NO: 2 - protein, 6 aa, synthetic construct
  REGION 1..6: note = synthetic peptide motif
  VARIANT 1: note = Xaa is any amino acid
  VARIANT 6: note = Xaa is any amino acid
XCGPCX

SEQ ID NO: 3 - protein, 6 aa, synthetic construct
  REGION 1..6: note = synthetic peptide motif
WCGPCK

SEQ ID NO: 4 - protein, 109 aa, Pseudomonas syringae
MSNDLIKHVTDASFEADVLKADGAVLVDYWAEWCGPCKMIAPVLDEIATTYAGKLTIAKLNIDENQETPAKHGVRGIPTLMLFKNGNVEATKVGALSKSQLAAFLDANI

SEQ ID NO: 5 - protein, 104 aa, Porphyromonas gingivalis
MALQITDATFDGLVAEGKPMVVDFWATWCGPCRMVGPIIDELAAEYEGRAIIGKVDVDANTELPMKYGVRNIPTILFIKNGEVVKKLVGAQSKDVFKKELDALF

SEQ ID NO: 6 - protein, 103 aa, Listeria monocytogenes
MVKEITDATFEQETSEGLVLTDFWATWCGPCRMVAPVLEEIQEERGEALKIVKMDVDENPETPGSFGVMSIPTLLIKKDGEVVETIIGYRPKEELDEVINKYV

SEQ ID NO: 7 - protein, 103 aa, Saccharomyces cerevisiae
MVTQFKTASEFDSAIAQDKLVVVDFYATWCGPCKMIAPMIEKFSEQYPQADFYKLDVDELGDVAQKNEVSAMPTLLLFKNGKEVAKVVGANPAAIKQAIAANA

SEQ ID NO: 8 - protein, 105 aa, Gallus gallus
MVKSVGNLADFEAELKAAGEKLVVVDFSATWCGPCKMIKPFFHSLCDKFGDVVFIEIDVDDAQDVATHCDVKCMPTFQFYKNGKKVQEFSGANKEKLEETIKSLV

SEQ ID NO: 9 - protein, 105 aa, Mus musculus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 10 - protein, 105 aa, Rattus norvegicus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEATITEFA

SEQ ID NO: 11 - protein, 105 aa, Bos taurus
MVKQIESKYAFQEALNSAGEKLVVVDFSATWCGPCKMIKPFFHSLSEKYSNVVFLEVDVDDCQDVAAECEVKCMPTFQFFKKGQKVGEFSGANKEKLEATINELI

SEQ ID NO: 12 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 13 - protein, 134 aa, Arabidopsis thaliana
MGGALSTVFGSGEDAAAAGTESSEPSRVLKFSSSARWQLHFNEIKESNKLLVVDFSASWCGPCRMIEPAIHAMADKFNDVDFVKLDVDELPDVAKEFNVTAMPTFVLVKRGKEIERIIGAKKDELEKKVSKLRA

SEQ ID NO: 14 - protein, 167 aa, Zea mays
MAMETCFRAWALHAPAGSKDRLLVGNLVLPSKRALAPLSVGRVATRRPRHVCQSKNAVDEVVVADEKNWDGLVMACETPVLVEFWAPWCGPCRMIAPVIDELAKDYAGKITCCKVNTDDSPNVASTYGIRSIPTVLIFKGGEKKESVIGAVPKSTLTTLIDKYIGSS

SEQ ID NO: 15 - protein, 172 aa, Oryza sativa
MALETCFRAWATLHAPQPPSSGGSRDRLLLSGAGSSQSKPRLSVASPSPLRPASRFACQCSNVVDEVVVADEKNWDSMVLGSEAPVLVEFWAPWCGPCRMIAPVIDELAKEYVGKIKCCKVNTDDSPNIATNYGIRSIPTVLMFKNGEKKESVIGAVPKTTLATIIDKYVSS

SEQ ID NO: 16-21 - skipped (6 entries)

SEQ ID NO: 22 - protein, 6 aa, synthetic construct
  REGION 1..6: note = synthetic peptide motif
  VARIANT 1: note = Xaa = any amino acid other than cysteine
  VARIANT 6: note = Xaa = any amino acid other than cysteine
XCGPCX

SEQ ID NO: 23 - protein, 6 aa, synthetic construct
  REGION 1..6: note = synthetic peptide motif
  VARIANT 5: note = Xaa = any amino acid other than cysteine
WCGPXK

SEQ ID NO: 24-25 - skipped (2 entries)

SEQ ID NO: 26 - protein, 6 aa, synthetic construct
  REGION 1..6: note = synthetic peptide motif
  VARIANT 1: note = Xaa = any amino acid other than cysteine
  VARIANT 6: note = Xaa = any amino acid other than cysteine
XCGPSX

SEQ ID NO: 27 - protein, 6 aa, synthetic construct
  REGION 1..6: note = synthetic peptide motif
WCGPSK

SEQ ID NO: 28 - protein, 105 aa, synthetic construct
  REGION 1..105: note = synthetic peptide motif
  VARIANT 96: note = any amino acid other than lysine
  VARIANT 94: note = any amino acid other than lysine
  VARIANT 72: note = any amino acid other than lysine
MVKQIESKTAFQEALDAAGDKLVVVDFSATWCGPCKMIKPFFHSLSEKYSNVIFLEVDVDDCQDVASECEVXCMPTFQFFKKGQKVGEFSGANXEXLEATINELV

SEQ ID NO: 29 - protein, 105 aa, synthetic construct
  REGION 1..105: note = synthetic peptide motif
MVKQIESKTAFQEALDAAGDKLVVVDFSATWCGPCKMIKPFFHSLSEKYSNVIFLEVDVDDCQDVASECEVACMPTFQFFKKGQKVGEFSGANAEALEATINELV